(19) United States
(12) Patent Application Publication
(10) Pub. No.: US 2023/0253114 A1
(43) Pub. Date: Aug. 10, 2023

(54) UNIVERSAL MEDICAL DIAGNOSTIC SYSTEM

(71) Applicant: Ben F. Bruce, Arlington, TX (US)

(72) Inventor: Ben F. Bruce, Arlington, TX (US)

(21) Appl. No.: 18/300,259

(22) Filed: Apr. 13, 2023

Related U.S. Application Data
(63) Continuation-in-part of application No. 16/422,294, filed on May 24, 2019, now abandoned.
Application No. 16/422,294 is a continuation-in-part of application No. 14/094,579, filed on Dec. 2, 2013, now abandoned.
(60) Provisional application No. 61/797,206, filed on Dec. 3, 2012.

(51) Int. Cl. G16H 50/20; G16H 10/60; G16H 70/60; G16H 40/67

(52) U.S. Cl. CPC G16H 50/20; G16H 10/60; G16H 70/60; G16H 40/67

(57) ABSTRACT

A method for controlling operations of a computer system for providing medical diagnoses, comprises diagnosing a patient, wherein the diagnosing comprises receiving a patient identification of the patient; receiving electronic data indicating the geographic location of the computer system; receiving the patient's chief complaint and interview data; receiving electronic data indicating one or more current body characteristics of the patient comprising at least one of pulse rate, body temperature, blood pressure, respiration, and skin condition; creating a current multimedia representation for each of the one or more current body characteristics determined by using the one or more sensors; identifying, in the computer system, previous multimedia representations of each of the one or more body characteristics from other persons using one or more trained classifiers; comparing the current multimedia representation to the previous multimedia representations; and identifying potential matches with corresponding confidence factors in accordance with defined medical standards.

### RELATED APPLICATION(S)

This patent application claims the benefit of priority, under 35 U.S.C. Section 119(e), to U.S. Provisional Patent Application Ser. No. 61/797,206, filed on Dec. 3, 2012, which is incorporated herein by reference and is a continuation of U.S. patent application Ser. No. 16/422,294, entitled MEDICAL ANALYSIS AND DIAGNOSTIC SYSTEM, filed May 24, 2019, which is incorporated herein by reference.

### COPYRIGHT

A portion of the disclosure of this document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever. The following notice applies to the software, data, and/or screenshots which may be described below and in the drawings that form a part of this document: Copyright © 2022, Trinity Technical Group, Inc. All Rights Reserved.

### TECHNICAL FIELD

The present invention relates generally to the field of medical examination, evaluation, triage, diagnosis and treatment, and more particularly to a method, system and program for making specific and unambiguous, or high confidence informed decisions on the diagnosis of medical and/or trauma conditions without prior knowledge, prescreening or referrals of what illness, malady, disease, infection, condition, allergic reaction or trauma is afflicting the patient. The World Health Organization (WHO) currently estimates that there are in excess of 10,000 diseases worldwide with 6,000-7,000 considered rare and the National Institute of Health (NIH) says that only approximately 500 of those diseases have an approved US Food and Drug Administration (FDA) approved treatment. This presents a significant problem with respect to identifying and diagnosing even those with FDA approved treatment, and this is what the Universal Medical Diagnostic system (UMDS) is designed to address. There are many medical diagnostic systems in existence, but they are almost exclusively designed to verify or confirm the presence of a specific disease or condition that is already known or suspected. The UMDS requires no prior knowledge, prescreening or referrals, but rather performs its diagnosis by first determining the geographic area in which it is located and then interviewing the patient and receiving the Chief Complaint along with other patient information and then using analog, digital and/or digitizing sensors, and inputs from various interfaces to gather patient information that is then processed, analyzed, classified, characterized, recognized and compared with historical patient data in trained classifiers to generate criteria suitable for use with one or more trained diagnostic engine(s). The geographic location is particularly important for a system such as UMDS that is capable of diagnosing medical conditions on a world-wide basis because many illnesses, maladies, diseases, infections or conditions exist in only limited geographic locations, e.g., polio is limited to only three countries; Ebola is limited to certain locations in sub-Saharan Africa; Lupus is limited to certain countries in the northern hemispheres, etc., etc. One or more expert systems, state machines, classifiers, regressors, neural networks or other methodologies are implemented as trained diagnostic engines and such trained diagnostic engines utilize all available criteria derived from the collected and processed patient data, vital signs, symptoms and historical data, if available, to select diagnostic templates while using a software algorithm to utilize the minimum number of tests required and if multiple candidates are produced, submit them to a trained arbitrator that will attempt to make a unique and unambiguous or high confidence diagnosis of an illness, malady, disease, infection, condition or trauma afflicting the patient. In the event that a unique and unambiguous or high confidence diagnosis cannot be made based upon the collected patient data, signs and symptoms, the system may recommend additional testing that will aid in producing a unique and unambiguous or high confidence diagnosis with as few additional tests as possible by utilizing the hierarchical pointers embedded in the diagnostic templates during their generation process. In the event that the diagnosis remains ambiguous, the system may refer the patient to a medical doctor or specialist along with a list of candidate diagnoses for further treatment. Once a diagnosis is finalized, the system will look up the recommended treatment regime associated with the diagnosis along with any associated prescription or non-prescription pharmaceuticals. Finally, the system will print off hard copies of the diagnosis and treatment regime as well as a list of any associated non-prescription pharmaceuticals and/or prescriptions for any prescription pharmaceuticals. The system will then save all current patient data into the patient's file for future reference, even if the data is not pertinent to the patient's current diagnosis. It should be noted that this system incorporates and utilizes approved US medical policies, standards, protocols, procedures and guidelines in the creation of the trained classifiers, trained diagnostic engines and trained arbitrators, as well as during the operation of the system. Furthermore, previously verified and approved patient data test sets may be used to test individual UMDS systems that will verify standardized, reliable, repeatable and accurate diagnostic and treatment results that are in accordance with those approved medical standards, protocols and guidelines.

### BACKGROUND

The approaches described in this section could be pursued but are not necessarily approaches that have been previously conceived or pursued. Therefore, unless otherwise indicated herein, the approaches described in this section are not prior art to the claims in this application and are not admitted to be prior art by inclusion in this section.

The collection of medical patient signs, symptoms and data; analysis of these signs, symptoms and data; diagnosis of medical conditions; and determination of curative treatment have traditionally been provided by medical doctors or specialists who have been through many years of specialized education, training and experience.

A number of devices are available to these medical doctors for use in collecting patient data which can be used to help them make a decision on a diagnosis of the specific illness, malady, disease, infection, condition or trauma afflicting the patient. Among other things, these devices may include scales, thermometers, stethoscopes, sphygmomanometers, and otoscopes. Once the patient's chief complaint has been identified and other patient information gathered, these devices can be used to collect pertinent patient signs, symptoms and data that the medical doctor or specialist may utilize, along with his or her own personal education, training, experience, memory and cognitive skills to make a decision on a diagnosis and recommend curative treatment regimens which may or may not include prescription or over-the-counter pharmaceuticals. If the doctor is unable to make a decision on a diagnosis, the patient may be referred for additional testing or to a medical specialist.

Additional laboratory testing may include, but is not limited to, blood tests, urinalysis, cultures, electrocardiogram (ECG or EKG), Sonogram/Ultrasounds, X-rays, Computerized Axial Tomography (CAT) Scans, Magnetic Resonance Imaging (MRI) or Positron Emission Tomography (PET) Scans may also be required in order to more definitively identify the illness, malady, disease, infection and/or trauma conditions affecting the patient.

Currently, notes related to patient data, examination, diagnosis, treatment and pharmaceuticals prescribed are often written by hand and copies, if any, are put into a patient file which is physically stored in the local facility. Some associated test results such as blood tests, urinalysis and electrocardiogram (ECG or EKG) may be printed out in hard copy and may be cross referenced to or included in the patient's file as well. Results of other tests such as Sonogram/Ultrasounds, X-rays, Computerized Axial Tomography (CAT) Scans, Magnetic Resonance Imaging (MRI) or Positron Emission Tomography (PET) Scans may be recorded in other media types and may be stored locally or in other facilities and may or may not be cross-referenced to the patient for future reference. Even digital copies of patient data, examinations, diagnoses, treatment and pharmaceuticals prescribed are often only shared within a limited network of hospitals and clinics unless copies are specifically requested by another attending physician or doctor. Furthermore, these digital copies are often not searchable in their stored format and must be physically read by attending physicians.

It should also be noted that not all doctors and physicians always operate in a standardized, repeatable, testable, consistent and documented manner, and this lack of standardization, repeatability and consistency on the part of doctors and physicians has led to a Johns Hopkins study released on May 3, 2016 that found medical errors are actually the third leading cause of death in the United States, after heart disease and cancer, and these errors include “ . . . unwarranted variation in physician practice patterns that lack accountability.”

With the foregoing in mind, UMDS is specifically designed to produce standardized, reliable, repeatable, accurate and testable diagnoses and associated treatment regimens that follow previously approved medical standards, protocols and guidelines. Furthermore, previously verified and approved patient data test sets are periodically used to test individual UMDS systems in order to demonstrate standardized, reliable, repeatable and accurate diagnostic and treatment results that are in accordance with approved US medical policies, standards, protocols, procedures and guidelines.

### SUMMARY

According to accepted and regularly annotated data from the World Health Organization and a study published by the University of Michigan Medical School, there are roughly 10,000 known diseases that afflict humans and the National Institute of Health (NIH) says that only approximately 500 of those diseases have an approved US Food and Drug Administration (FDA) approved treatment. This presents a significant problem with respect to identifying and diagnosing even those with FDA approved treatment, and this is what the Universal Medical Diagnostic system (UMDS) is designed to address. In 2019 alone there were approximately 58 million deaths worldwide attributable to communicable diseases, non-communicable diseases and trauma. Then there is the shortage of trained medical professionals and even within the United States, there are many areas that have no doctors at all or that are considered medically underserved. As of Dec. 31, 2022, according to the US Department of Health and Human Services (DHHS) there were approximately 8,294 designated Primary Care Health Professional Shortage Areas (HPSAs) within the US. To be more specific, the State of Texas has 254 counties, many of which have no medical doctor at all and 154 of which have been designated by the US DHHS as being HPSAs. This situation is not expected to improve in the foreseeable future and will arguably get worse as increasing numbers of medical doctors move into more lucrative specialties. Finally, there are the natural disasters that occur both in the US and around the world that overwhelm the local health care community. Just one case in point is Hurricane Katrina, where the hurricane and subsequent flooding killed over 1,833 people, injured thousands more and displaced hundreds of thousands in addition to that. Organized health care suffered a systemic collapse, hospitals and clinics were evacuated and health care providers effectively ceased to be available for tens of thousands of people in the Greater New Orleans area. In other areas around the world there are hurricanes, typhoons, cyclones, tornados, earthquakes, tsunamis and floods that kill and injure thousands of people nearly every year, and leave thousands of others displaced and without adequate medical care, and that does not include all those people in underdeveloped countries, war zones, refugee camps, remote areas, etc. UMDS is designed for use in all those locations and scenarios to test for and diagnose all known communicable diseases and trauma, and many non-communicable diseases that have been adequately documented, and do this without any prior knowledge, prescreening or referrals prior to testing and making a diagnosis. Instead, UMDS starts each session as tabula rasa and provides its diagnosis based upon its geographic location, the patient's Chief Complaint and interview, their present vital signs and symptoms, and in some cases, their personal or family history. In some example embodiments, a method for controlling operations of a computer system for providing medical diagnoses includes diagnosing a patient in accordance with approved US medical policies, standards, protocols, procedures and guidelines; the diagnosing includes knowing the geographic location in which it is located; receiving a patient identification of the patient; receiving patient's Chief Complaint and interview; the diagnosing includes determining, using digital or digitizing sensors, the current body characteristics of the patient comprising at least, but not limited to the geographic area, biological sex, race, age, pulse rate, body temperature, blood pressure, respiration rate, skin condition, breathing sounds and other characteristics contained in the templates; the diagnosing includes creating a current digital or multimedia representation for each of the one or more current body characteristics determined by using the sensors and; the diagnosing includes using a trained classifier to compare the current multimedia representation to previous multimedia representations of each of the body characteristics from the same or other persons and produce matching results along with corresponding confidence factors for each multimedia representation. UMDS then feeds all of the resulting digital and multimedia representations, their associated characteristics and confidence factors into one or more trained diagnostic engines. The diagnosing includes using trained diagnostic engine(s) with a closely coupled diagnostic template database containing diagnostic templates of all known and documented illnesses, maladies, diseases, infections, conditions, allergic reactions or traumas along with all their known geographic locations, characteristics, associated signs and symptoms, the weighting factor for each sign or symptom and the high confidence factor threshold required for a diagnosis. The diagnostic engines then use repetitive comparisons with the diagnostic templates in accordance with a least number of tests algorithm to select one or more diagnoses and diagnosis confidence factors for the patient based on the comparing of the current digital and/or multimedia representation to the previous digital and/or multimedia representations of each of one or more body characteristics. The trained diagnostic engines then feed their resulting diagnoses and cumulative diagnosis confidence factors to a trained arbitrator that selects the best diagnosis based upon approved US medical policies, standards, protocols, procedures and guidelines. The diagnosing includes determining whether the diagnosis confidence factor exceeds an acceptable threshold, known as the high confidence factor threshold, assigned to each diagnostic template. The diagnosing includes in response to the diagnosis confidence factor not exceeding the high confidence factor threshold, selecting a different current body characteristic of the patient in accordance with the least number of tests algorithm to increase the diagnosis confidence factor. The diagnosing includes in response to the diagnosis confidence factor exceeding the high confidence factor threshold, selecting the diagnosis for the patient.

### BRIEF DESCRIPTION OF THE DRAWINGS

The embodiments are provided by way of example and not limitation in the figures of the accompanying drawings, in which like references indicate similar elements and in which:

FIG. 1 is a system diagram for a Universal Medical Diagnostic System, according to some example embodiments.

FIG. 2 is a system diagram for possible use in a standalone mobile or facility environment, according to some example embodiments.

FIG. 3 is a system diagram for possible use in a facility or remote distributed (client/server) environment, according to some example embodiments.

FIG. 4 is a system diagram for possible use in a facility or remote distributed (client/server) environment, according to some example embodiments.

FIG. 5 is a system diagram for possible use in a facility for offline creation of trained classifiers, trained diagnostic engines and trained arbitrators, according to some example embodiments.

FIG. 6 is a diagram of a method for offline training of a classifier component in a Universal Medical Diagnostic System, according to some example embodiments.

FIG. 7 is a diagram of a method for offline training of a diagnostic engine component in a Universal Medical Diagnostic System, according to some example embodiments.

FIG. 8 is a diagram of a method for offline training of an arbitrator component in a Universal Medical Diagnostic System mode, according to some example embodiments.

FIG. 9 is a diagram of a method for main processing in a Universal Medical Diagnostic System, according to some example embodiments.

FIG. 10 is a diagram of a method for a diagnostic mode in a Universal Medical Diagnostic System, according to some example embodiments.

FIG. 11 is a diagram of a method for a monitor mode in a Universal Medical Diagnostic System, according to some example embodiments.

FIG. 12 is a diagram of a method for a physical examination mode in a Universal Medical Diagnostic System, according to some example embodiments.

FIG. 13 is a diagram of a method for a treatment determination mode in a Universal Medical Diagnostic System, according to some example embodiments.

FIG. 14 is a diagram of a method for a continuation of the diagnostic mode in a Universal Medical Diagnostic System, according to some example embodiments.

FIG. 15 is a diagram of a method for a maintenance mode in a Universal Medical Diagnostic System, according to some example embodiments.

FIG. 16 is a diagram of a method for a sensor operation and diagnostic verification mode in a Universal Medical Diagnostic System, according to some example embodiments.

FIG. 17 is a diagram of a method for the classifier portion of a diagnostic mode in a Universal Medical Diagnostic System, according to some example embodiments.

FIG. 18 is a diagram of a method for the diagnostic engine portion of a diagnostic mode in a Universal Medical Diagnostic System, according to some example embodiments.

FIG. 19 is a diagram of a method for the arbitrator portion of a diagnostic mode in a Universal Medical Diagnostic System, according to some example embodiments.

### DETAILED DESCRIPTION

Methods, apparatus and systems for a Universal Medical Diagnostic System (UMDS) are described. In the following description, numerous specific details are set forth. However, it is understood that embodiments of the invention may be practiced without these specific details. In other instances, structures and techniques have not been shown in detail in order not to obscure the understanding of this description.

Some example embodiments of UMDS may utilize a mobile computer system with specialized hardware, firmware and databases, and may include a basic sensor suite such as, but not limited to analog, digital or digitizing sensors such as scales, stethoscopes, thermometers, sphygmomanometers, perfusion oxygen or hematocrit saturation monitors, ophthalmoscopes, funduscopes, and otoscopes to gather patient information that may include but not be limited to weight, height, pulse rate, pulse characterization and pattern recognition, respiration rate, respiration and body sounds characterization and pattern recognition, body temperature, blood pressure, oxygen saturation, perfusion, skin temperature, skin moisture level, electrocardiogram, imaging and/or video of eyes, ears, nose and throat, and imaging and/or video for skin, scalp and extremities to collect data to be transmitted to and processed by the mobile system. Such sensors are capable of collecting analog, digital, discrete, pressure, audio, high definition color and/or grayscale image and video, and/or other data types and converting this multimedia data to a format suitable for uploading to the UMDS for use in the classification and diagnostic evaluation processes. UMDS includes at least one trained classifier/diagnostic engine/arbitrator component. This trained classifier/diagnostic engine/arbitrator component may consist of expert systems, state machines, classifiers, regressors, neural networks or other methodologies that may be implemented as one or more trained classifier/diagnostic engine components and such trained classifier/diagnostic engine components may each be configured or tuned differently, but processed in parallel and their results adjudicated by the trained arbitrator in order to emulate a team of different doctors evaluating the same patient data in order to classify diagnostic hypotheses as either being correct or in error. It does this by using a very large number of characteristics associated with each element of a diagnostic hypothesis resulting from the evaluation process of individual patient data sources such as audio, video, image, pattern or other data types related to both normal conditions as well as all known illnesses, maladies, diseases, infections, conditions or traumas and evaluates them to discover patterns that are highly correlated to either correct or erroneous diagnostic hypotheses. These patterns are also cross-validated offline, during the training process of the classifier/diagnostic engine/arbitrator component, to measure their predictive diagnostic performance on a large blind data set of patient body characteristics whose correct diagnostic results are known a priori.

The classifier/diagnostic engine/arbitrator requires this training to be done a priori. During the training process, the classifier performs advanced analysis, called inductive machine learning, of the capabilities, strengths and weaknesses of all the characteristics resulting from the evaluation process of each individual patient body characteristics in multimedia formats such as audio, video, image, pattern or other data types and uses the results of that analysis as part of the process of building decision trees or other machine learning models designed to minimize or eliminate errors and maximize successful diagnoses. This evaluation process utilizes large volumes of previously examined and validated samples of each multimedia data type representing examples of every known illness, malady, disease, infection, condition or trauma associated with that multimedia data type, as well as normal conditions for those multimedia data types. The process of developing and validating the factors used in creating accurate and precise diagnostic templates (also known as machine learning models) is an offline, automated process that is very computationally intensive, but the result of this offline process is a set of decision trees or other machine learning models that are very fast to use when generating elements for online diagnostic templates. One or more classifier components may be incorporated that evaluate each patient body characteristic, create multimedia representations and produce corresponding results along with confidence factors for each multimedia representation. All of the resulting multimedia representations, their associated characteristics and confidence factors are then fed into one or more trained diagnostic engines for use in generating diagnostic templates. A secondary classifier component may be incorporated that may be utilized to compare current subject patient data samples with the subject patient's own historical data samples from prior diagnostic or examination sessions on the subject patient and evaluate each sample from each data type and produce corresponding results along with confidence factors for each data type. All of the resulting data types, their associated characteristics and confidence factors may then be fed into one or more trained diagnostic engines. It is understood that these data types may represent both normal and abnormal conditions. A diagnostic database that contains all known and documented illnesses, maladies, diseases, infections, conditions or traumas and their associated geographic area, Chief Complaint, race, biological sex, height, weight, complaints, signs, symptoms and vital signs as well as normal conditions is used by the trained diagnostic engine component during the development of evaluation processes and diagnostic templates during offline training of the diagnostic engine. A medical standards, protocols and guidelines database that contains all of the most current applicable and approved medical standards, protocols and guidelines associated with diagnosing each illness, malady, disease, infection, condition or trauma is also utilized in order to ensure that each diagnostic hypothesis complies with all of the most current applicable and approved medical standards, protocols and guidelines in order to minimize or eliminate potential misdiagnosis or other diagnostic errors. This database of medical standards, protocols and guidelines is used by the trained diagnostic engine component in the development of evaluation processes and diagnostic templates during offline training of the diagnostic engine. It should be noted that the diagnostic database also includes weighted symptom values, which are the weighted values assigned to each symptom based upon the specific disease, illness, malady, infection, condition, allergic reaction or trauma that the symptom is associated with. This is to say that if a range of diagnoses has the same value symptom, those symptoms will all have the same weighted symptom value regardless of the measured symptom value. If, however, the symptom is more significant or central in some diagnoses than in others, then that symptom may be assigned a higher weighted symptom value than in other cases based upon the measured symptom value. For example, a high temperature symptom is more likely in a diagnosis of Strep throat than with simply a sore throat and therefore a high temperature symptom would have a higher weighted symptom value in a diagnosis of Strep; certain lung (breathing) sounds symptoms are more likely in some diagnoses than others, e.g., wheezing in the case of bronchitis or asthma, rhonchi in the case of mucus buildup in the large airways, stridor in the case of epiglottitis or croup, or rales in the case of pulmonary edema and pneumonia. Therefore, those lung (breathing) sounds symptoms would be assigned a higher weighted symptom value in those diagnoses. These weighted symptom values are all added to the cumulative diagnostic value and compared with the high confidence diagnosis values. If the cumulative diagnostic value meets or exceeds the high confidence diagnosis value, then that diagnosis should be selected. High confidence diagnosis values are assigned to each specific disease, illness, malady, infection, condition, allergic reaction or trauma in accordance with the most current applicable and approved medical standards, protocols and guidelines in order to select the diagnosis that has the highest probability of being the correct diagnosis when compared to all other potential diagnoses. A high confidence informed decision on a diagnosis is one where the cumulative diagnostic value meets or exceeds the high confidence diagnosis value for that diagnosis. The diagnostic engine component also requires training a priori that is a very CPU intensive process. It involves utilizing the diagnostic database and the most current applicable and approved medical standards, protocols and guidelines in order to identify geographic area, Chief Complaint, race, biological sex, height, weight, complaints, signs, symptoms and vital signs associated with each known illness, malady, disease, infection, condition, allergic reaction or trauma as well as normal conditions and running a full regression analysis on any diagnostic hypothesis resulting from the evaluation of each patient data set. References and pointers to required complaints, signs, symptoms and vital signs are also generated and included in the diagnostic templates in order to allow specific tests associated with those complaints, signs, symptoms and vital signs to be requested in order to minimize the amount of testing required. A diagnostic template is generated for each illness, malady, disease, infection, condition, allergic reaction or trauma in the diagnostic database. This process determines which factors or characteristics provide an indication that the elements of the diagnostic template corresponding to those factors or characteristics is correct when compared to other patient data sets with correct and validated diagnosis results known a priori and that they are in accordance with all of the most current applicable and approved medical standards, protocols and guidelines, and a confidence factor is generated that corresponds to the correlation for factors and characteristics when compared to other patient data sets with correct and validated diagnosis results known a priori. The diagnostic engine may also determine that a diagnostic template requires additional information from the patient and what information is required to move forward with determining or finalizing a diagnostic template. If this required information is not already available, the diagnostic engine will return a request for the required information (e.g., a throat swab to determine strep throat or a nose swab to determine influenza) based upon the references and pointers for that information included in the diagnostic template. A completed diagnostic template may represent both normal and abnormal conditions, and these completed diagnostic templates are stored in the diagnostic template database.

The trained arbitrator component also requires training a priori that is a very CPU intensive process. It involves utilizing the known characteristics of each trained classifier and trained diagnostic engine along with the diagnostic templates and their associated confidence factors generated by each trained classifier/diagnostic engine component and utilizing that information along with the approved medical standards, protocols and guidelines to select the one that has the highest probability of being the correct diagnostic hypothesis.

During the online diagnostic evaluation process, UMDS will evaluate samples from each data source collected from a patient by using trained classifiers to evaluate each sample from each data type and produce matching results along with corresponding confidence factors for each data type. UMDS will then feed all of the resulting data types, their associated characteristics and confidence factors into one or more trained diagnostic engines. Using the diagnostic templates created during the offline training process, the trained diagnostic engines will utilize one or more of the evaluated data types, representing either normal or abnormal conditions and corresponding to the patient's Chief Complaint, to attempt to complete one or more diagnostic templates and will generate confidence factors based upon the completed elements of the diagnostic template and the diagnostic values associated with those completed elements. The trained diagnostic engines may also utilize historical patient data and diagnoses for comparison with patient's current geographic area, Chief Complaint, race, biological sex, height, weight, complaints, signs, symptoms and vital signs to generate higher confidence factors for the diagnostic templates.

The following methodology represents one of many potential methods of utilizing the least number of tests to determine a unique and unambiguous diagnosis or high confidence informed decision on a diagnosis. Other methodologies may be implemented in parallel in order to maximize the probability of determining a unique and unambiguous diagnosis or high confidence informed decision on a diagnosis. The first step in utilizing the least number of tests to determine a unique and unambiguous diagnosis or high confidence informed decision on a diagnosis is to receive the geographic location and then perform the patient interview including determining the Chief Complaint and acquiring answers to all patient interview questions and storing the results. The second step is to acquire and store all patient medical data using the basic sensor suite regardless of its potential relationship to the patient's current Chief Complaint or answers to the patient interview questions, and initializing the cumulative diagnostic value to zero. The third step is to initiate an initial search of the Diagnostic Template Database using the diagnostic engine(s), looking for potential diagnosis results, using as search criteria the patient's Chief Complaint and associated primary search values, complaints, signs, symptoms and vital signs in order to establish an initial pool of all potential disease, illness, malady, infection, condition, allergic reaction or trauma candidates. As the individual patient symptoms are matched against symptoms in the Diagnostic Template Database, their weighted symptom values are added to the cumulative diagnostic value for comparison against the high confidence diagnosis value. If a unique and unambiguous diagnosis or high confidence informed decision on a diagnosis with no additional mandatory test(s) is found, then select such diagnosis or high confidence informed decision as the final diagnosis. If there are any matching high confidence informed decision diagnosis results that include pointers to additional mandatory test(s) then perform those mandatory tests. If the mandatory tests are negative, select the original highest confidence diagnosis results as the high confidence diagnosis. If the mandatory tests are positive, select the mandatory test results as the high confidence diagnosis. If the High Confidence threshold is not met, then the next step is to select the most common symptom in the established pool of potential diseases, illnesses, maladies, infections, conditions, allergic reactions or trauma candidates and run the patient's symptom results against the resulting pool of candidates. If a unique and unambiguous diagnosis or high confidence informed decision on a diagnosis with no additional mandatory test(s) is found, then select such diagnosis or high confidence informed decision as the final diagnosis. If there are any matching high confidence informed decision diagnosis results that include pointers to additional mandatory test(s) then perform those mandatory tests. If the mandatory tests are negative, select the original highest confidence diagnosis results as the high confidence diagnosis. If the mandatory tests are positive, select that diagnosis as the high confidence diagnosis. If a unique and unambiguous diagnosis or high confidence informed decision on a diagnosis is not found in that group of candidate diagnoses, then eliminate that group of candidate diagnoses from the established pool and repeat the foregoing step with the remaining pool until a unique and unambiguous diagnosis or high confidence informed decision on a diagnosis is identified or all candidate diagnoses are eliminated from the pool. If all candidate diagnoses are eliminated from the pool without identifying a unique and unambiguous diagnosis or high confidence informed decision on a diagnosis, refer the patient to a medical doctor or specialist for further treatment. If multiple candidate diagnoses are found, the resulting diagnostic templates along with their confidence factors will be fed into the trained arbitrator and the trained arbitrator will process the diagnostic templates and their associated confidence factors generated by each trained classifier/diagnostic engine component and utilize that information to select the one that has the highest probability of being the correct diagnostic hypothesis. In some cases, the trained arbitrator may also search the remote patient database for other patients with similar complaints, signs, symptoms and vital signs, and their resulting diagnostic hypotheses for comparison purposes in order to reach a higher confidence factor for the patient's diagnostic hypotheses. If the trained arbitrator returns a diagnostic decision or diagnosis, it will also assign a confidence factor generated by the matching algorithms and return this confidence factor for each diagnosis based upon the completeness and significance of the patient's geographic location, Chief Complaint, race, biological sex, height, weight, complaints, signs, symptoms and vital signs. This confidence factor, typically a percentage between 0 and 100, will be indicative of the confidence that the trained arbitrator has of each selected diagnosis. The trained arbitrator may also determine that a potential diagnosis requires additional information from the patient and what information is required to move forward with determining a diagnosis. If this required information is not already available, the trained arbitrators will return a request for the required information. In the event that a unique and unambiguous diagnosis or a high confidence decision on a diagnosis cannot be made based upon the available patient geographic location, Chief Complaint, race, biological sex, height, weight, complaints, signs, symptoms and vital signs, the UMDS may recommend additional testing that will aid in producing a unique and unambiguous diagnosis or a high confidence decision on a diagnosis with as few tests as possible, based upon the factors and characteristics of the diagnostic template or templates in use. In the event that the diagnosis remains ambiguous or has a low confidence factor, the UMDS may refer the patient to a medical doctor or specialist for further treatment. The definition of a numeric value for a high confidence factor threshold for UMDS will be determined by the appropriate medical authorities. If a unique and unambiguous diagnosis or a high confidence diagnosis is determined, UMDS will look up the recommended treatment regimen associated with the diagnosis along with any associated prescription or non-prescription pharmaceuticals. Finally, the system may print off hard copies of the diagnosis and treatment regimen along with a list of any associated non-prescription pharmaceuticals and/or prescriptions for any prescription pharmaceuticals. The system will then save all current patient data into the patient's file for future reference.

Some example embodiments may utilize a mobile computer system with specialized hardware, firmware, software and/or databases, and an associated sensor suite for collection of patient geographic location, Chief Complaint, race, biological sex, height, weight, complaints, signs, symptoms and vital signs for processing by UMDS as described in the foregoing paragraphs. This system will be capable of functioning in a standalone mobile or facility environment and when connected to LAN, WAN, wireless, cellular or other network services, such mobile systems will be able to download and utilize any existing and available remote patient databases or historical patient files along with patient digital discrete, pressure, image, video, audio or other media inputs or files from results from more sophisticated laboratory and test equipment such as, but not limited to, blood tests, urinalysis, cultures, x-ray machines, contact or non-contact tonometry, Sonogram/Ultrasound, Electrocardiogram, Computerized Axial Tomography (CAT) scans, Magnetic Resonance Imaging (MRI) and Positron Emission Tomography (PET) scans for further processing.

Other example embodiments might include dedicated or client/server systems in fixed locations that are capable of servicing multiple clients in one or more local or remote locations. Such systems may utilize specialized hardware, firmware, software and/or databases in the server systems while the client systems may utilize associated sensor suite for collection of patient geographic location, Chief Complaint, race, biological sex, height, weight, complaints, signs, symptoms and vital signs that are passed to the server systems for processing by the UMDS as described in the foregoing paragraphs.

Other example embodiments might include a touch screen, keyboard or other manual inputs for operator identification and verification, patient name and personal information, medical information including, but not limited to age, height, weight, known conditions, known drug allergies, current prescriptions, etc., and other information as required. Touch screen, keyboard or other manual inputs may also be used to input the Chief Complaint(s) and input answers to predetermined lists of questions based upon whether the patient has a trauma or is suffering from a medical condition. Finally, touch screen, keyboard or other manual inputs may be utilized to enter manual results or operator observed results including but not limited to rebound tenderness, swelling, joint swelling, joint displacement, etc.

Other example embodiments of the present invention might include specialized audio processing, image processing, video processing and other multimedia processing types that may be used along with image, audio, video, pattern and/or other multimedia recognition algorithms, all of which may be implemented in hardware, firmware, software or any combination thereof.

Other example embodiments might include processing, analyzing, classifying, recognizing, characterizing and/or comparing any available analog, digital, discrete, pressure, image, video, audio or other media inputs by hardware, firmware or software to identify any signs, symptoms, potential anomalies or abnormal characteristics and produce criteria suitable for use in the trained diagnostic engines.

Other example embodiments might include processing, analyzing, classifying, recognizing, characterizing and comparing any currently available and/or historical inputs or other media files such as, but not limited to age, sex, body weight, pulse rate, respiration rate, body temperature, blood pressure, oxygen saturation, skin temperature and moisture level, and perfusion being processed, analyzed, classified, correlated, recognized, characterized and/or compared in order to identify any vital signs, symptoms, potential anomalies or abnormal characteristics and produce criteria suitable for use in the trained diagnostic engines.

Other example embodiments might include currently available and/or historical audio, pressure or other inputs or media files being processed, analyzed, classified, correlated, recognized, characterized and/or compared with respect to heartbeat characterization and pattern recognition, pulse characterization and pattern recognition, respiration, breathing and other body sounds in order to identify any signs, symptoms, potential anomalies or abnormal characteristics and produce criteria suitable for use in the trained diagnostic engines.

Other example embodiments might include currently available and historical image or video inputs or other media files being processed, analyzed, classified, recognized, characterized and/or compared with respect to signs or symptoms including but not limited to pupil size and relative pupil size; pupil reaction to light; eye conditions including, but not limited to conjunctivitis (pink eye), uveitis, iritis, scleritis, keratitis and stye (bump on the eye); ear canal and ear drum; nasal passages; throat; skin medical conditions including, but not limited to rashes, blisters, ulcers, acne, eczema, ringworm, psoriasis, scabies, shingles, psoriasis, rosacea, basal cell carcinoma, squamous cell carcinoma, and melanoma; skin trauma conditions including, but not limited to contusions (bruises), abrasions (scrapes), lacerations (cuts, scratches or punctures), burns (chemical or heat); serious skin trauma conditions; nail conditions including, but not limited to hangnail, fungus, ingrown nail; scalp or hair conditions including, but not limited to alopecia, head lice, dandruff, ingrown hair; and any other items of interest such as, but not limited to swellings, joint swelling or joint displacement; internal medical conditions including but not limited to tumors, growths, cysts, cancers, aneurysms, hernias, broken or dislocated bones and any other medical issues in order to identify any signs, symptoms, potential anomalies or abnormal characteristics and produce criteria suitable for use in the online trained diagnostic engines.

Other example embodiments might include currently available discrete, pressure, image, video, audio or other inputs or media files being processed, analyzed, classified, recognized, characterized, compared and correlated with historical discrete, image, video, audio or other media files to do a comparative analysis in order to identify any differences, signs, symptoms, potential anomalies, abnormal characteristics and/or trends, and produce criteria suitable for use in the trained diagnostic engines.

Other example embodiments might include the implementation of a diagnostic search engine or engines as expert systems, state machines, classifiers, regressors, neural networks or other methodologies that utilize currently available geographic and point in time information as part of the search criteria submitted to the trained diagnostic engines.

Another example embodiment of the present invention provides a methodology wherein if a unique and unambiguous diagnosis or a high confidence decision on a diagnosis cannot be obtained with the available patient information and data, the online trained arbitrator should produce a list of possible diagnoses with confidence factors for each one and based upon the current circumstances and available patient data, and provide this list to a medical doctor or specialist for further treatment.

Another illustrated embodiment of the present invention provides a methodology to standardize patient interviews, data collection, diagnostics, treatment regimens and dispensing of prescriptions according to approved medical standards, protocols and guidelines.

Another illustrated embodiment of the present invention provides a methodology for sharing remote patient medical information via cellular, wireless, Local Area Network (LAN), Wide Area Network (WAN) or other connectivity and using that information from different sources to improve the patient's diagnostic results and resulting health care.

Another illustrated embodiment of the present invention provides a methodology for implementing a trained arbitrator component to utilize the known characteristics of each trained classifier/diagnostic engine component along with the diagnoses and their associated confidence factors generated by each trained classifier/diagnostic engine component and utilizing that information to select the one that has the highest probability of being the correct diagnostic hypothesis.

Another illustrated embodiment of the present invention provides a methodology for processing, analyzing, classifying, correlating, recognizing, characterizing and/or comparing multiple patients' complaints, signs, symptoms, vital signs, allergic reactions and/or diagnoses based on geographic areas to determine if there is a potential for related medical issues in specific geographic areas (e.g., outbreaks, epidemics, Ebola, Lyme Disease, Legionnaires Disease, etc.).

Another illustrated embodiment of the present invention provides a methodology for processing, analyzing, classifying, correlating, recognizing, characterizing and/or comparing multiple patients' complaints, signs, symptoms, vital signs, and/or diagnoses to determine if there is a potential for related medical issues in patients with similar physicality, physiology, race, ethnicity, gender, work, background, familial relations, environment, geographic location, medical diagnoses, marital status, etc.

Another illustrated embodiment of the present invention provides the ability to reprogram or update the configuration and/or tuning parameters for the classifier/diagnostic engine/arbitrator software or firmware system wide using encrypted data and controlled software approval and release methodologies.

Another illustrated embodiment of the present invention provides the ability to update diagnostic, treatment and pharmaceutical databases; medical standards, protocols and guidelines; and search algorithms system wide using encrypted data and controlled software approval and release methodologies.

Another illustrated embodiment of the present invention provides a methodology for storing patient data and utilizing both currently available and historical patient data in making a diagnosis or in identifying trends that may be detrimental to the health of the patient.

Another illustrated embodiment of the present invention provides a methodology for processing, analyzing, classifying, correlating, recognizing, characterizing and/or comparing heartbeat, pulse data and/or breathing sounds or other data to identify signs, symptoms, latent or potential anomalies, abnormal characteristics and/or trends that may require further investigation or treatment.

Another illustrated embodiment of the present invention provides a method for continuously monitoring patient sensor data while the patient is being treated, transported or is under care in a facility, hospital, emergency room or Intensive Care Unit (ICU) and continuously evaluating the patient's condition based upon the collected and analyzed data. Should the patient's data exceed approved medical standards, the system would take predetermined actions including alerting on-duty medical personnel.

Another example embodiment of the present invention allows the injection of previously verified and approved patient data test sets into individual UMDS systems that will demonstrate standardized, reliable, repeatable and accurate diagnostic and treatment results that are in accordance with approved medical standards, protocols and guidelines.

Another illustrated embodiment of the present invention provides a methodology for using a Certified Self Test Unit (CSTU) to ensure that the basic sensor suite is correctly calibrated and all sensors are reading within specified parameters.

Such embodiments are in contrast to conventional techniques for identifying, diagnosing and treating the illness, malady, disease, infection, condition or trauma afflicting the patient. In particular, using conventional techniques, identifying, diagnosing and treating illnesses, diseases, infections or trauma must be done by or under the direction or supervision of licensed and certified medical doctors or specialists, whereas these embodiments may utilize a trained operator without the participation, supervision or intervention of a medical doctor or specialist.

A more detailed description of the systems, apparatus and methods for gathering, processing, analyzing, classifying, recognizing, characterizing and/or comparing patient data and utilizing the results to make a unique and unambiguous or a high confidence decision on a diagnosis and the associated treatment regimen is now described.

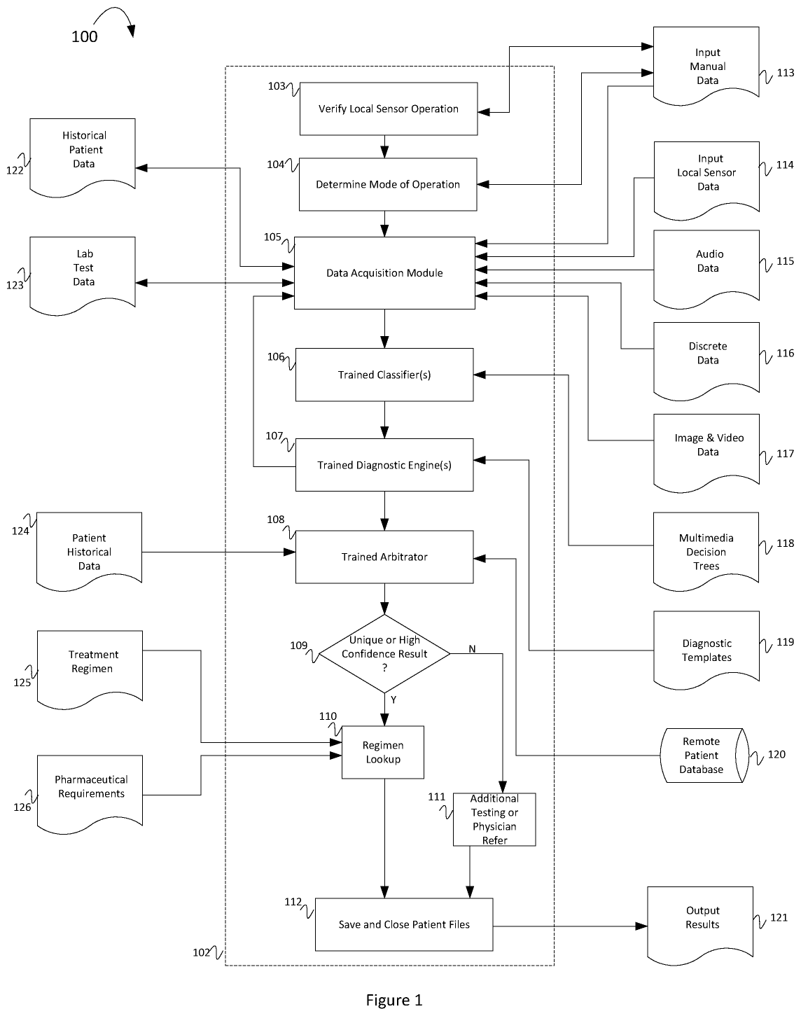

FIG. 1 is a system diagram for a universal medical diagnostic system, according to some example embodiments. FIG. 1 illustrates a system 100 that includes a universal medical diagnostic system. The universal medical diagnostic system 102 may be a mobile system or a fixed base client/server system serving both local and remote systems. In some example embodiments, the universal medical diagnostic system 102 may operate in a semi-autonomous manner without being directly connected to additional laboratory test equipment. In other example embodiments, the universal medical diagnostic system 102 may operate in a semi-autonomous manner and may or may not be directly connected to additional laboratory test equipment. Moreover, as further stated below, the various modules of the medical analysis and diagnostic system may all reside within a single processing unit.

Universal medical diagnostic system 102 comprises a sensor verification module 103, a mode of operation module 104, a data acquisition module 105, trained classifier(s) 106, trained diagnostic engine(s) 107, a trained arbitrator 108, a regimen lookup module 110 and a data retention module 112. Mode of operation 104 receives manual inputs 113 to identify and verify the operator, determine the mode of operation and uniquely identify the patient. Data acquisition module 105 receives additional manual inputs 113 to provide unique identification of the patient, chief complaint(s) and other patient information, local sensor data 114, audio data 115, discrete data 116, image & video data 117, historical patient data 122, if available, and lab test data 123, if requested and available. Data acquisition module 105 will then pass the collected data on to the trained classifier(s) 106 for further processing. The trained classifier(s) 106 will process, analyze, classify, correlate, characterize, recognize and/or compare local sensor data 114, audio data 115, discrete data 116, image and video data 117 and any other data types, files and media collected from the manual inputs 113, historical patient data 122 and lab test data 123 as it becomes available and process it in accordance with the decision trees or other machine learning models 118 to produce data and confidence factors for use in the trained diagnostic engine 107. It is understood that the trained classifier(s) 106 may consist of hardware, software and/or firmware components or a mixture thereof. The trained diagnostic engine(s) 107 may consist of one or more expert systems, state machines, classifiers, regressors, neural networks or other methodologies and utilizes all available data including currently available geographic and point in time information, patient chief complaint(s), patient interviews, processed patient sensor data with confidence factors, processed patient data with confidence factors and any available patient historical data to complete all applicable previously created diagnostic templates with associated confidence factors.

If the trained diagnostic engine(s) 107 is able to identify a unique and unambiguous diagnosis, then this diagnosis will be passed to the trained arbitrator. Otherwise, if a high confidence decision on a diagnosis can be made, then this diagnosis will be passed to the trained arbitrator. If the diagnosis is ambiguous and does not exceed the high confidence threshold, then the diagnostic engine 107 will utilize the diagnostic template(s) to determine additional tests to remove the ambiguity and/or increase the confidence factor and pass this information back to the data acquisition module 105. Once an unambiguous or high confidence diagnosis is identified, the diagnostic engine 107 will pass that information to the trained arbitrator 108. The trained arbitrator 108 will utilize all diagnostic results with confidence factors, all known classifier characteristics, any patient historical data sets with known correct diagnostic results 124, the remote patient database 120 and all medical guidelines, protocols and standards to evaluate the diagnostic results and select the final diagnosis. The final diagnosis will then be tested for being unique and unambiguous or for a high confidence result 109. If the final diagnosis is unique and unambiguous or a high confidence result, it will be passed on the regimen lookup module 110, which will identify the corresponding treatment regimen 125 and any associated pharmaceutical requirements 126. The regimen lookup module 110 will then pass the final diagnosis, the corresponding treatment regimen and any associated pharmaceutical requirements to output results 121 to be made available to the operator and/or the patient. Save and close patient files 112 is then accomplished and the analysis and diagnostic session is ended.

Operations, according to example embodiments, are now described. In certain embodiments, the operations are performed by instructions residing on machine-readable media (e.g., software or firmware), while in other embodiments, the methods are performed by hardware or other logic (e.g., digital logic).

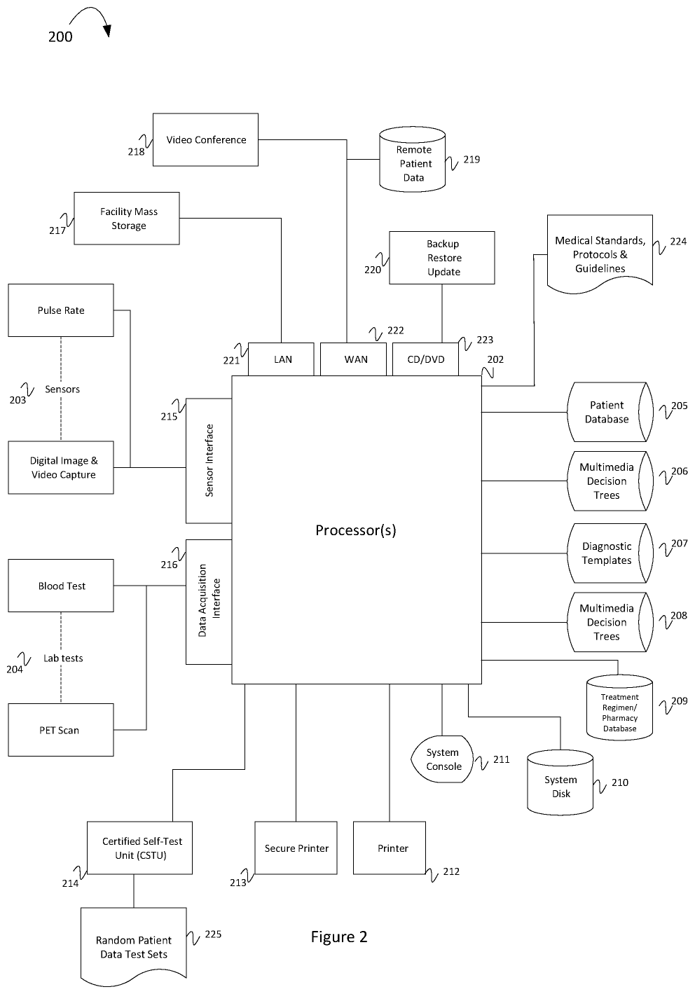

FIG. 2 is a detailed block diagram for a computer system that controls operations of system for providing semiautonomous universal medical diagnoses, according to some example embodiments, and is now described. In particular, FIG. 2 illustrates for a computer system that controls operations of system for providing universal medical diagnoses that may be used in a standalone mobile or facility environment, according to some example embodiments. As illustrated in FIG. 2, the computer system 200 comprises processor(s) 202 which also includes any necessary memory, internal bus, input/output controllers, various interfaces, one or more disk drive(s), one or more database(s), storage facilities, sensors, network connections, printers, console(s) and a certified self-test unit. The processor(s) 202 may comprise any suitable processor architecture. The computer system that controls operations of the semiautonomous universal medical diagnostic system 200 may comprise one, two, three, or more processors, any of which may execute a set of instructions in accordance with embodiments of the invention.

Various local analog, digital or digitizing sensors 203 are utilized to collect analog, digital, discrete, pressure, audio, high definition color and/or grayscale image and video, and/or other data types and convert this data to a format suitable for uploading to the mobile computer system through interface 215 for further processing, analyzing, classifying, correlating, characterizing, pattern recognition and/or comparing, by the trained classifier(s) and generating data and associated confidence factors suitable for use by the trained diagnostic engine(s), according to some example embodiments.

Laboratory test equipment 204 may or may not be connected through interface 216 to download analog, digital, discrete, pressure, audio, image and/or video data, and other data types, files and media as they become available for further processing, analyzing, classifying, correlating, characterizing and/or pattern recognition, comparing and generation of search criteria suitable for use with the diagnostic search engine. It will be understood by those skilled in the art that interfaces 215 and 216 may be implemented using LAN, WAN, USB, Bluetooth, wireless, cellular, proprietary or other network communication protocols, or a combination thereof in order to maximize connectivity, efficiency and throughput, according to some example embodiments.

According to some example embodiments, one or more databases may be implemented to provide access to required information. Patient database 205 will contain all available local data and files on the patient currently being examined or treated. The diagnostic database 206 will contain the most currently available medical information on all known illnesses, diseases, infections, traumas allergic reactions and other maladies. The diagnostic template database 207 will contain diagnostic templates utilized by the diagnostic engine(s) to determine potential diagnoses and their associated confidence factors. Database 209 will contain the most currently available recommended treatment regimens associated with the illnesses, diseases, infections, traumas and other maladies contained in the diagnostic database 206, including whether over-the-counter or prescription pharmaceuticals are indicated as part of the treatment regimen. If so, the pharmacy database includes the most currently available list of over-the-counter and prescription pharmaceuticals and if they are indicated as part of the treatment regimen, the patient's digital folder or record will be accessed to determine if there are any known redundancies, drug reactions, allergies or potential interactions with other prescribed medications and whether any of the patient's current symptoms represent known side effects of the patient's current medications. The approved medical guidelines, protocols and standards database 224 will be utilized during training of the classifiers and diagnostic engines, and will be utilized during performance testing of the UMDS.

The remote patient database 219 will contain all patient data, and associated complaints, signs, symptoms and vital signs for all patients that have been examined by UMDS along with the final diagnoses and confidence factors. It will be understood by those skilled in the art that two or more of these databases may be consolidated into a single database.

The system console 211 may be a console, keyboard, touch screen or other manual input device and is used for system dialog and maintenance functions, as well as a data acquisition module to input manual inputs to provide unique identification of the patient, chief complaint(s) and other patient information. System disk 210 holds all operating system and application software, according to some example embodiments. Printer 212 may be used to print off patient information, diagnosis, treatment regimens, pharmaceuticals and any other required information, according to some example embodiments. Secure printer 213 is utilized to print off prescriptions and other secure documents as required, according to some example embodiments.

It will be understood by those skilled in the art that interfaces 221, 222 and 223 may be implemented using LAN, WAN, USB, Bluetooth, wireless, cellular, proprietary or other network communication protocols, or a combination thereof in order to maximize connectivity, efficiency and throughput, and may be connected to remote patient data files 219, backup, restore or update 220, facility mass storage 217, and/or allow for video conferencing 218, according to some example embodiments.

A certified self-test unit 214 may be implemented in order to ensure that the local sensor suite is correctly calibrated and all sensors are reading within specified parameters, according to some example embodiments. Random patient data test sets with known results 225 may be injected into the UMDS to ensure that the system produces the correct diagnoses for the random patient data test sets, according to some example embodiments.

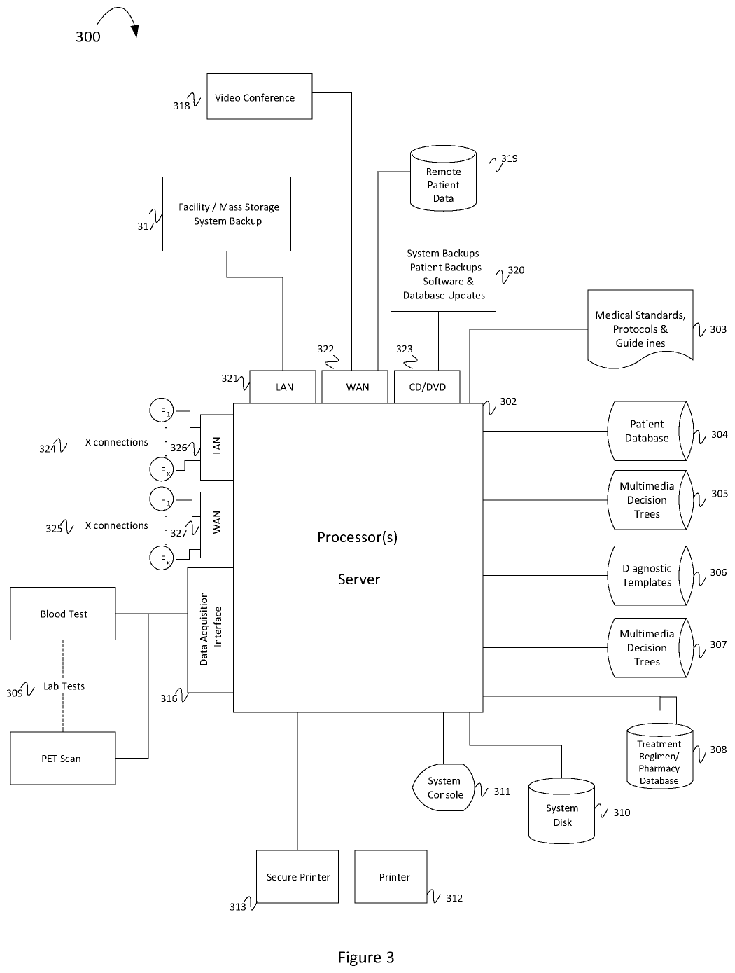

FIG. 3 is a detailed block diagram for a method for controlling operations of a computer system in a semiautonomous universal medical diagnostic system, according to some example embodiments, and is now described. In particular, FIG. 3 illustrates a method for controlling operations of a computer system in a semiautonomous universal medical diagnostic system that may be used as the server in a facility or remote distributed (client/server) environment, according to some example embodiments. As illustrated in FIG. 3, the computer system 300 comprises processor(s) 302 which also includes any necessary memory, internal bus, input/output controllers, various interfaces, one or more disk drive(s), one or more database(s), storage facilities, sensors, network connections, printers, console(s) and a self-test unit. The processor(s) 302 may comprise any suitable processor architecture. The computer(s) controlling the operations of the semiautonomous universal medical diagnostic system 300 may comprise one, two, three, or more processors, any of which may execute a set of instructions in accordance with embodiments of the invention.

Multiple local or remote client systems 324 and 325 may be connected to the server through interfaces 326 and 327 for downloading client sensor analog, digital, discrete, pressure, audio, high definition color and/or grayscale image and/or video, and/or other data types for further processing, analyzing, classifying, characterizing, pattern recognition and/or comparing by the trained classifier(s) and generating data and associated confidence factors suitable for use by the trained diagnostic engine(s), according to example embodiments.

Laboratory test equipment 309 may or may not be connected through interface 316 to download analog, digital, discrete, audio, pressure, image and/or video data, and/or other data types, files and media as they become available for further processing, analyzing, classifying, characterizing, pattern recognition and/or comparing, and generating of search criteria suitable for use with the diagnostic search engine. It will be understood by those skilled in the art that interfaces 316, 326 and 327 may be implemented using LAN, WAN, USB, Bluetooth, wireless, cellular, proprietary or other network communication protocols, or a combination thereof in order to maximize connectivity, efficiency and throughput, according to some example embodiments.

According to some example embodiments, one or more databases may be implemented to provide access to required information. Patient database 304 will contain all available data and files on the patient currently being examined or treated. The diagnostic database 305 will contain the most currently available medical information on all known illnesses, diseases, infections, traumas and other maladies. The diagnostic template database 306 will contain diagnostic templates utilized by the diagnostic engine(s) to determine potential diagnoses and their associated confidence factors. Database 308 will contain the most currently available recommended treatment regimens associated with the illnesses, diseases, infections, traumas and other maladies contained in the diagnostic database 305, including whether over-the-counter or prescription pharmaceuticals are indicated as part of the treatment regimen. If so, the pharmacy database includes the most currently available list of over-the-counter and prescription pharmaceuticals and if they are indicated as part of the treatment regimen, the patient's digital folder or record will be accessed to determine if there are any known redundancies, drug reactions, allergies or potential interactions with other prescribed medications and whether any of the patient's current symptoms represent known side effects of the patient's current medications. The approved medical guidelines, protocols and standards database 303 will be utilized during training of the classifiers and diagnostic engines, and will be utilized during performance testing of the UMDS. The approved medical standards, protocols and guidelines database 303 will be utilized during training of the classifiers and diagnostic engines, and will be utilized during performance testing of the UMDS. It will be understood by those skilled in the art that two or more of these databases may be consolidated into a single database.

After the diagnostic session is complete, any results, including required patient information, diagnosis, treatment regimens, pharmaceuticals and any other information is passed back to the appropriate local or remote client system 324 or 325 through interface 326 or 327, according to some example embodiments.

The system console 311 may be a console, keyboard, touch screen or other manual input device and is used for system dialog and maintenance functions, as well as a data acquisition module to input manual inputs to provide unique identification of the patient, chief complaint(s) and other patient information. System disk 310 holds all operating system and application software, according to some example embodiments. Printer 312 may be used to print off patient information, diagnosis, treatment regimens and any other required information, according to some example embodiments. Secure printer 313 is utilized to print off prescriptions and other secure documents as required, according to some example embodiments.

It will be understood by those skilled in the art that interfaces 321, 322 and 323 may be implemented using LAN, WAN, USB, Bluetooth, wireless, cellular, proprietary or other network communication protocols, or a combination thereof in order to maximize connectivity, efficiency and throughput, and may be connected to remote patient data files 319, backup, restore or update 320 facility mass storage 317, or allow for video conferencing 318, according to some example embodiments.

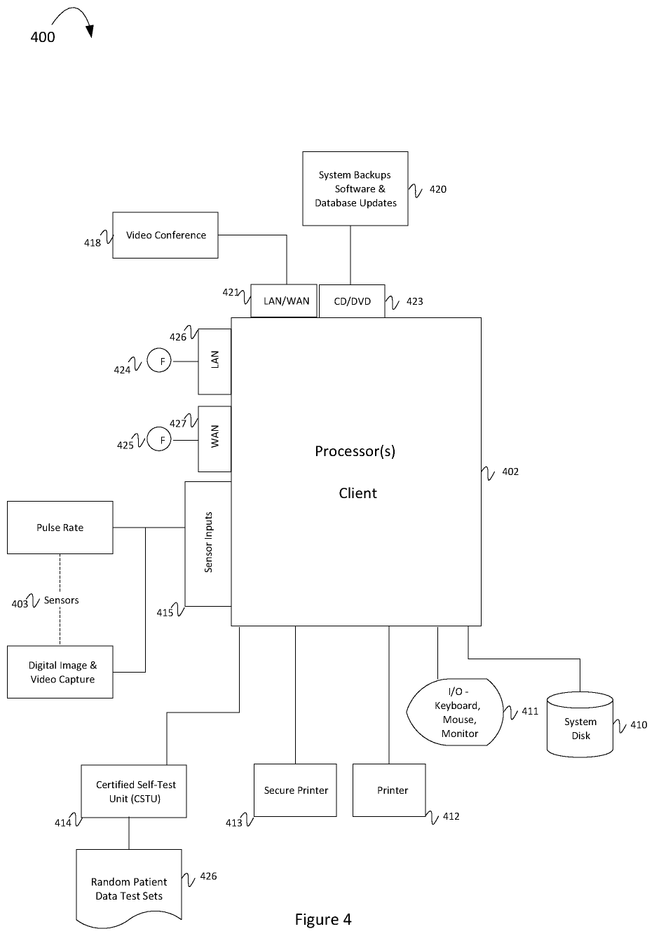

FIG. 4 is a detailed block diagram for a computer system that controls operations of a semiautonomous universal medical diagnostic system, according to some example embodiments, and is now described. In particular, FIG. 4 illustrates a computer system that controls operations of a semiautonomous universal medical diagnostic system that may be used as the client in a facility or remote distributed (client/server) environment, according to some example embodiments. As illustrated in FIG. 4, the computer system 400 comprises processor(s) 402 which also includes any necessary memory, internal bus, input/output controllers, various interfaces, one or more disk drive(s), one or more database(s), storage facilities, sensors, network connections, printers, console(s) and a self-test unit. The processor(s) 402 may comprise any suitable processor architecture. The computer(s) controlling the operations of the semiautonomous universal medical diagnostic system 400 may comprise one, two, three, or more processors, any of which may execute a set of instructions in accordance with embodiments of the invention.

According to some sample embodiments, various analog, digital or digitizing sensors 403 may be utilized to collect analog, digital, discrete, audio, pressure, image, video and/or other data types and converting this data to a format suitable for uploading to the client computer system through interface 415 for further processing, analyzing, classifying, characterizing, pattern recognition and/or comparing by the trained classifier(s) and generating data and associated confidence factors suitable for use by the trained diagnostic engine(s), according to some example embodiments.

The client system may be connected to server 424 or 425 through interface 426 or 427 for uploading client sensor analog, digital, discrete, audio, pressure, high definition color and/or grayscale image and video and/or other data types to the server for further processing, analyzing, classifying, characterizing, pattern recognition and/or comparing, and generation of search criteria suitable for use with the diagnostic search engine, according to example embodiments. It will be understood by those skilled in the art that interfaces 415, 426 and 427 may be implemented using LAN, WAN, USB, Bluetooth, wireless, cellular, proprietary or other network communication protocols, or a combination thereof in order to maximize connectivity, efficiency and throughput, according to some example embodiments.

After the diagnostic process is complete, any required patient information, diagnosis, treatment regimens, pharmaceuticals and any other required information is downloaded from the server back to the appropriate local or remote client system 424 or 425 through interface 426 or 427, according to some example embodiments.

The system console 411 may be a console, keyboard, touch screen or other manual input device and is used for system dialog and maintenance functions, as well as a data acquisition module to input manual inputs to provide unique identification of the patient, chief complaint(s) and other patient information. System disk 410 holds all operating system and application software, according to some example embodiments. Printer 412 may be used to print off patient information, diagnosis, treatment regimens and any other required information, according to some example embodiments. Secure printer 413 is utilized to print off prescriptions and other secure documents as required, according to some example embodiments.

It will be understood by those skilled in the art that interfaces 421 and 423 may be implemented using LAN, WAN, USB, Bluetooth, wireless, cellular, proprietary or other network communication protocols, or a combination thereof in order to maximize connectivity, efficiency and throughput, and may be connected to backup, restore or update 420 or allow for video conferencing 418, according to some example embodiments.

A certified self-test unit 414 may be implemented in order to ensure that the basic sensor suite is correctly calibrated and all sensors are reading within specified parameters, according to some example embodiments. Random patient data test sets with known results 426 may be injected into the UMDS to test the system operation and ensure that the system produces the correct diagnoses for the random patient data test sets, according to some example embodiments.

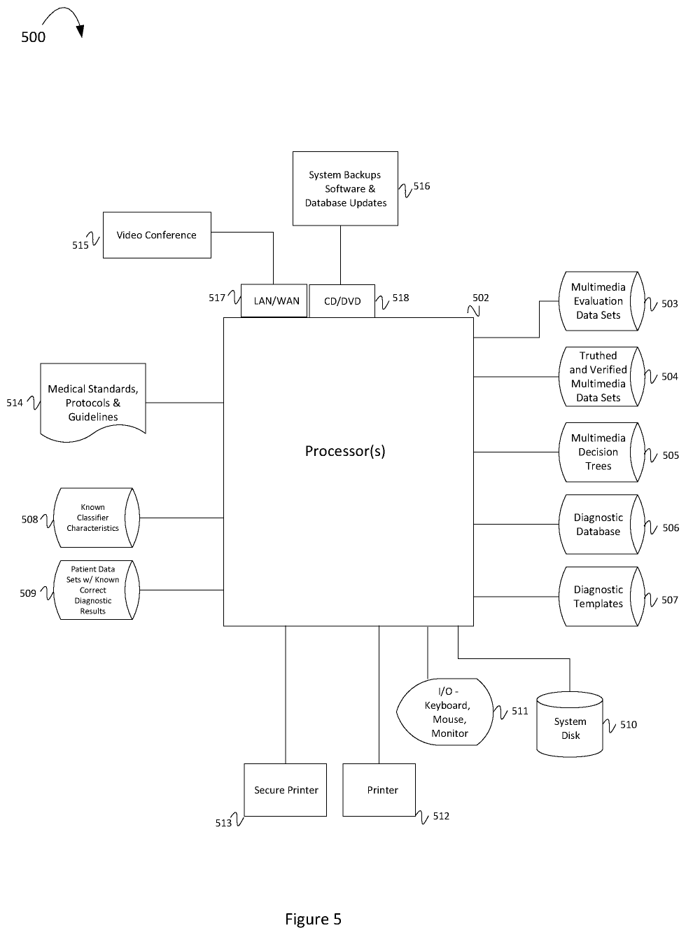

FIG. 5 is a detailed block diagram for a computerized offline classifier, diagnostic engine and arbitrator training system according to some example embodiments, and is now described. In particular, FIG. 5 illustrates a computerized system that may be used to train classifiers, diagnostic engines and arbitrators as part of the universal medical diagnostic system, according to some example embodiments.

As illustrated in FIG. 5, the computer system 500 comprises processor(s) 502 which also includes any necessary memory, internal bus, input/output controllers, various interfaces, one or more disk drive(s), one or more database(s), storage facilities, sensors, network connections, printers, console(s) and a self-test unit. The processor(s) 502 may comprise any suitable processor architecture. The computerized offline classifier, diagnostic engine and arbitrator training system 500 may comprise one, two, three, or more processors, any of which may execute a set of instructions in accordance with some embodiments of the invention.

Multimedia Evaluation Data Sets 503 and Truthed and Verified Multimedia Data Sets 504 are used by the offline classifier training system to create the Multimedia Decision Trees or other Machine Learning Models 505 in accordance with some embodiments of the invention.

A Diagnostic Database 506 and Medical Standards, Protocols & Guidelines 514 are used by the offline diagnostic engine training system to create the Diagnostic Templates 507 in accordance with some embodiments of the invention.

Known Classifier Characteristics 508, Medical Standards, Protocols & Guidelines 514 and Patient Data Sets w/Known Correct Diagnostic Results 509 are used by the offline arbitrator training system to create trained arbitrators in accordance with some example embodiments.

The system console 511 may be a console, keyboard, touch screen or other manual input device and is used for system dialog and maintenance functions. System disk 510 holds all operating system and application software, according to some example embodiments. Printer 512 may be used to print off any required information, according to some example embodiments. Secure printer 513 is utilized to print off any secure documents as required, according to some example embodiments.

It will be understood by those skilled in the art that interfaces 517 and 518 may be implemented using LAN, WAN, USB, Bluetooth, wireless, cellular, proprietary or other network communication protocols, or a combination thereof in order to maximize connectivity, efficiency and throughput, and may be connected to backup, restore or update 516 or allow for video conferencing 515, according to some example embodiments.

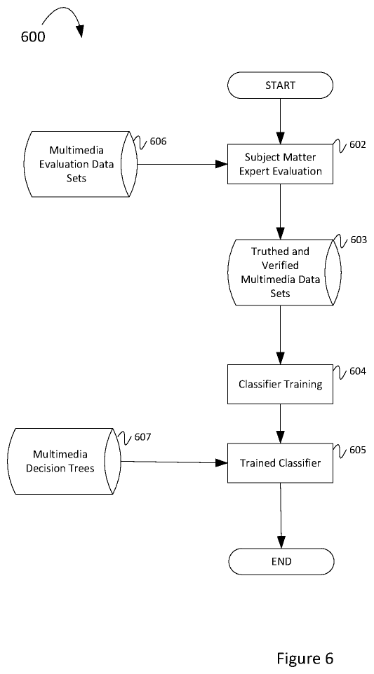

A method 600 is described with reference to FIG. 6. In some sample embodiments, FIG. 6 is a diagram of a method for training a multimedia classifier for use with a universal medical diagnostic system that includes block 602 where a subject matter expert evaluates multimedia evaluation data sets from 606 and the truthed and verified multimedia data sets (both true and false) are passed to 603 to be used in training the classifier(s) 604. During the training process, the classifier performs advanced analysis, called inductive machine learning, of the capabilities, strengths and weaknesses of all the characteristics resulting from the evaluation process of each individual patient body characteristics in multimedia formats such as audio, video, image, pattern or other data types and uses the results of that analysis as part of the process of building decision trees or other machine learning models designed to minimize or eliminate errors and maximize successful diagnoses. This evaluation process utilizes large volumes of previously examined and validated samples of each multimedia data type representing examples of every known illness, malady, disease, infection, condition or trauma associated with that multimedia data type, as well as normal conditions for those multimedia data types. The process of developing and validating the factors used in creating accurate and precise diagnostic templates is an offline, automated process that is very computationally intensive, but the result of this offline process is a set of decision trees or other machine learning models that are very fast to use when generating elements for online diagnostic templates. One or more classifier components may be incorporated that evaluate each patient body characteristic, create multimedia representations and produce corresponding results along with confidence factors for each multimedia representation. The trained classifier 605 is then able to utilize the multimedia decision trees or other machine learning models 607 when it is evaluating and classifying a patient's multimedia data types.

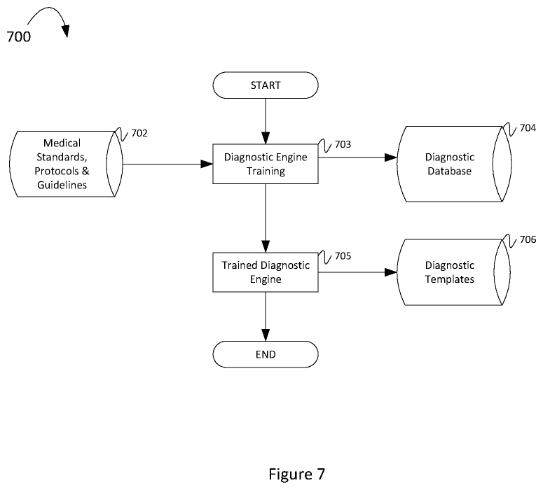

A method 700 is described with reference to FIG. 7. In some sample embodiments, FIG. 7 is a diagram of a method for training a diagnostic engine for use with a Universal Medical Diagnostic System that includes block 702 medical standards, protocols and guidelines associated with diagnosing each illness, malady, disease, infection, condition, allergic reaction or trauma along with 704 diagnostic database that contains all known illnesses, maladies, diseases, infections, conditions, allergic reactions or traumas and their associated complaints, signs, symptoms and vital signs as well as normal conditions. These are used during diagnostic engine training 703 while developing evaluation processes during offline training of the diagnostic engine that is a very CPU intensive process. It involves utilizing the diagnostic database 704 and the most current applicable and approved medical standards, protocols and guidelines 702 in order to identify complaints, signs, symptoms and vital signs associated with each known illness, malady, disease, infection, condition, allergic reaction or trauma as well as normal conditions and running a full regression analysis on any diagnostic hypothesis resulting from the evaluation of each patient data set. This process determines which factors or characteristics provide an indication that the elements of the diagnostic template 706 corresponding to those factors or characteristics is correct when compared to other patient data sets with correct and validated diagnosis results known a priori and that they are in accordance with all of the most current applicable and approved medical standards, protocols and guidelines, and a confidence factor is generated that corresponds to the correlation for factors and characteristics when compared to other patient data sets with correct and validated diagnosis results known a priori. The trained diagnostic engine 705 may also determine that a diagnostic template requires additional information from the patient and what information is required to move forward with determining or finalizing a diagnostic template. If this required information is not already available, the diagnostic engine will return a request for the required information (e.g., a throat swab to determine strep throat or a nose swab to determine influenza) based upon the references and pointers for that information included in the diagnostic template.

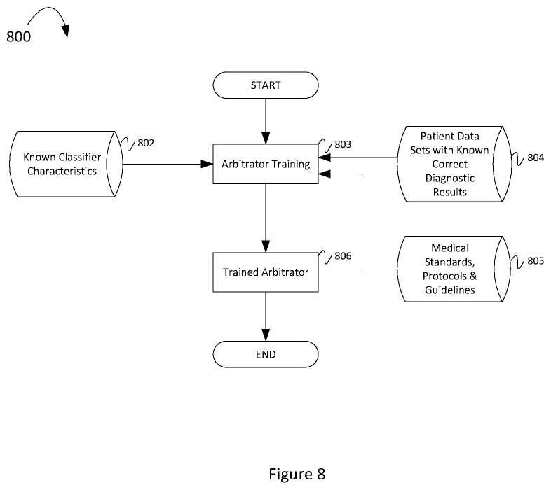

A method 800 is described with reference to FIG. 8. In some sample embodiments, FIG. 8 is a diagram of a method for creating a trained multimedia arbitrator for use with a universal medical diagnostic system that includes block 803 arbitrator training. The trained arbitrator requires training a priori that is a very CPU intensive process. It involves utilizing the known characteristics of each trained classifier 802, the medical standards, protocols and guidelines 805, and patient data sets with known correct diagnostic results 804 to be utilized to create a trained arbitrator 806 that utilizes all that information to select the completed diagnostic template that has the highest probability of being the correct diagnosis while complying with all approved medical standards, protocols and guidelines in order to minimize or eliminate diagnostic errors.

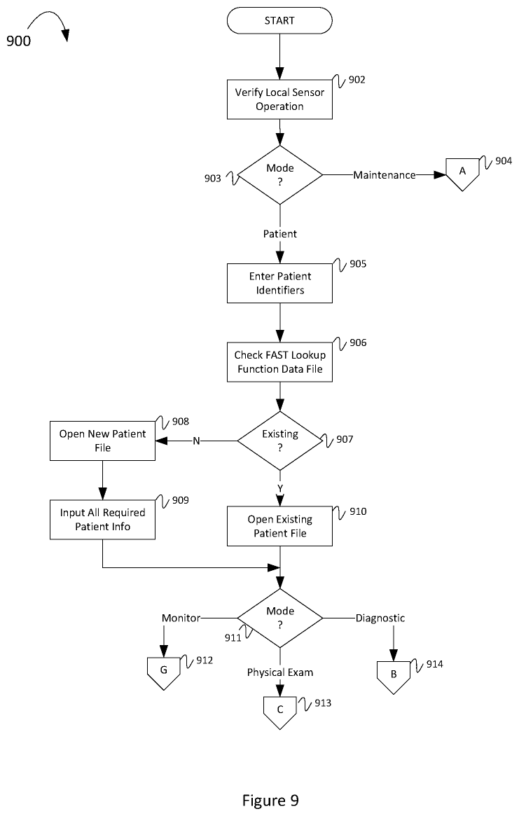

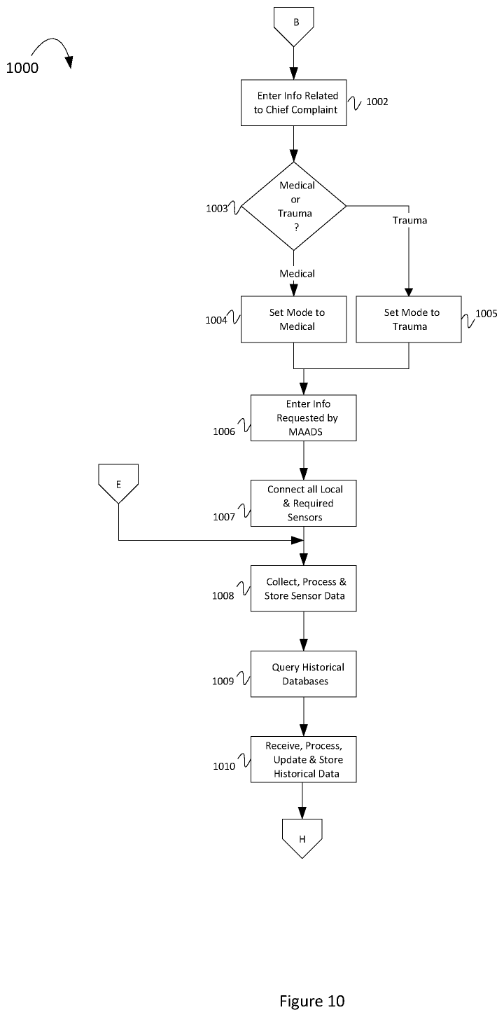

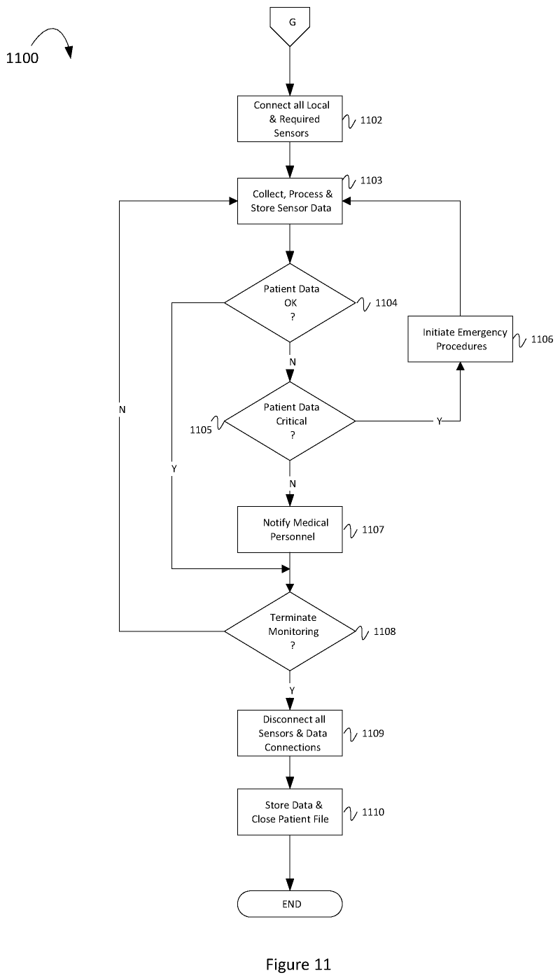

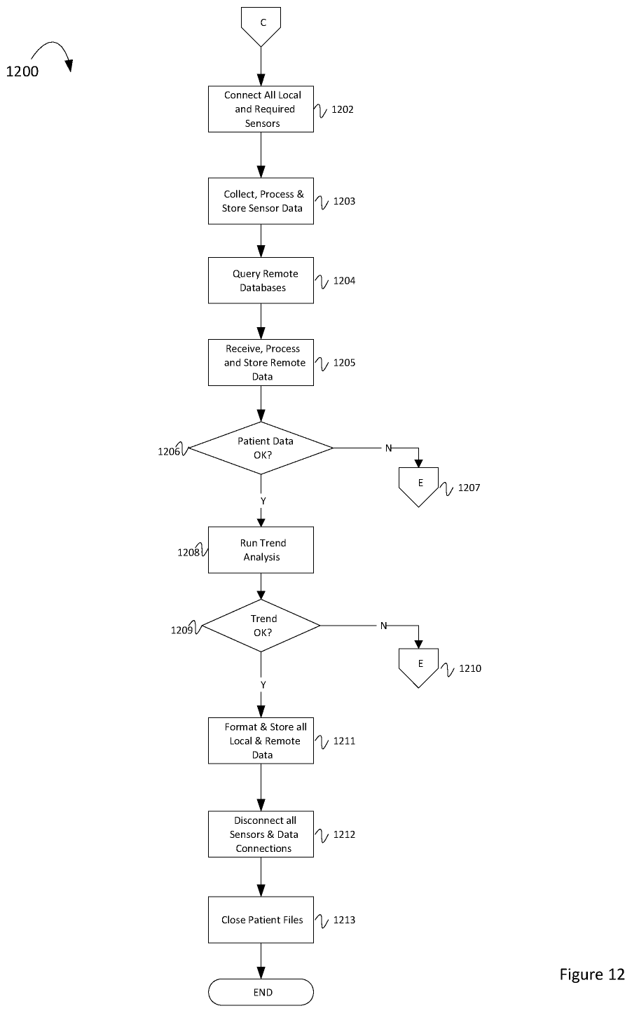

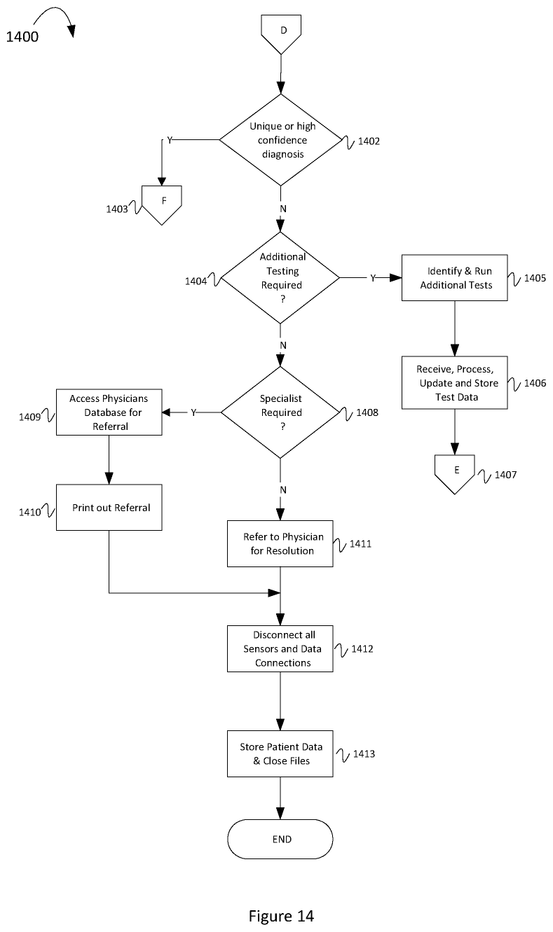

A method 900 is described with reference to FIG. 9. In some sample embodiments, FIG. 9 is a diagram of a method for a universal medical diagnostic system that includes block 902 for verifying local sensor operation; block 903 for determining the mode of operation as either maintenance or patient; if mode of operation is maintenance at block 903 then proceed to FIG. 14 (A) 904; if mode of operation is patient, then entering patient identifiers at block 905 to determine if this is a new or existing patient 907; either opening a new patient file at block 908 and populating it at block 909 or opening the existing patient file at block 910; determining the mode of operation at block 911 as either monitoring then proceed to FIG. 11 (G) 912, performing a physical examination then proceed to FIG. 12 (C) 913 or performing diagnostics then proceed to FIG. 10 (B) 914, according to some example embodiments.

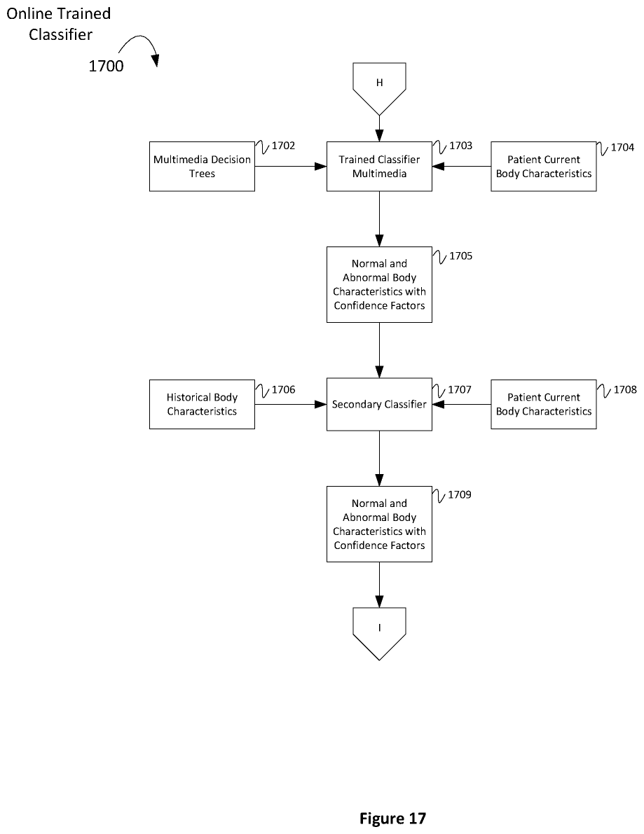

A method 1000 is described with reference to FIG. 10. In some sample embodiments, FIG. 10 is a diagram of a method for a universal medical diagnostic system diagnostic mode that includes acquiring patient information including geographic location, unique identification of the patient, chief complaint(s) 1002; determining whether the problem is medical or trauma related 1003 and setting the mode to medical 1004 or trauma 1005; performing the patient interview, updating or storing the patient information 1006; connecting all currently available local and required sensors to the patient 1007; collecting, processing and storing the currently available local sensor and laboratory test data 1008; querying patient historical databases 1009; locating, retrieving and processing historical data related to the patient 1010; proceeding to FIG. 17 (H) 1703 to continue diagnostic processing; according to some example embodiments.

A method 1100 is described with reference to FIG. 11. In some sample embodiments, FIG. 11 is a diagram of a method for a universal medical diagnostic system monitoring mode that includes connecting all local and required sensors 1102; collecting, processing, analyzing, classifying, comparing, recognizing, correlating, storing and/or comparing the currently available local sensor and laboratory test data 1103 to determine if patient data is within established parameters 1104 and, if so, check to see if monitoring is still required 1108; if patient data is outside parameters and critical, initiate emergency procedures 1106; if patient data is outside parameters and not critical, notify medical personnel 1107; if monitoring is no longer required 1108, disconnect all sensors and data connections 1109; store patient data and close patient files 1110, according to some example embodiments.

A method 1200 is described with reference to FIG. 12. In some sample embodiments, FIG. 12 is a diagram of a method for a universal medical diagnostic system physical examination mode that includes connecting all local and required sensors 1202; collecting, processing, analyzing, classifying, recognizing, comparing, storing and/or correlating the currently available local sensor and laboratory test data 1203; querying any remote databases 1204; receiving, processing, analyzing, classifying, recognizing, correlating and/or comparing and storing the local and remote data 1205; determining if patient data is within established parameters 1206 and if not within established parameters begin diagnostic mode 1207 at FIG. 9 (E); if patient data is okay then run a trend analysis 1208; if trend analysis is not okay 1209 then begin diagnostic mode 1210 at FIG. 9 (E); if trend analysis is okay then format and store all patient data 1211; disconnect all sensors and data connections 1212; and close patient files 1213, according to some example embodiments.

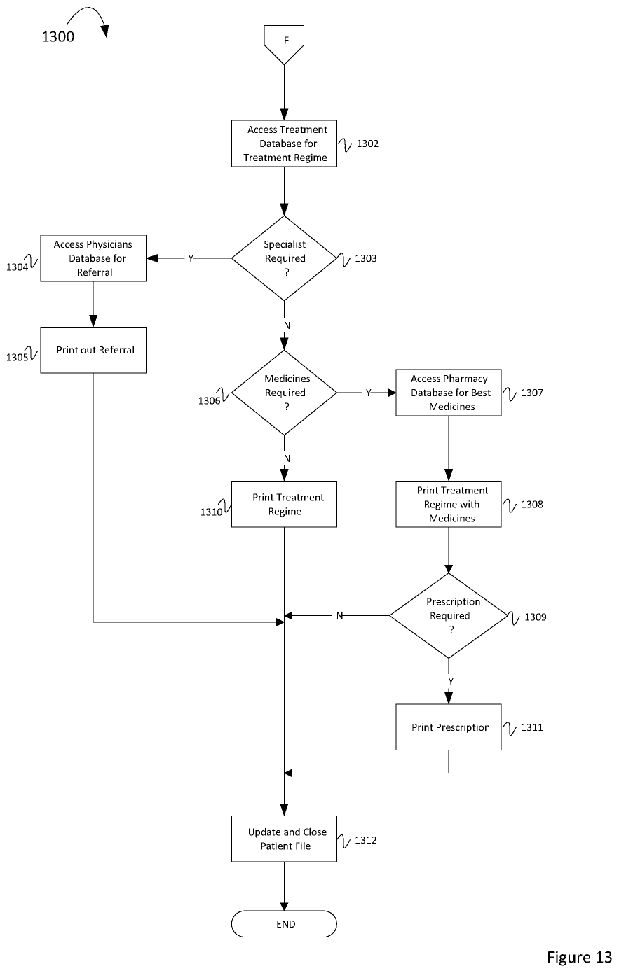

A method 1300 is described with reference to FIG. 13. In some sample embodiments, FIG. 13 is a diagram of a method for a universal medical diagnostic system treatment determination mode that includes accessing a treatment database 1302; determining if a medical specialist is required 1303 and if so, identifying a medical specialist 1304 and making a referral 1305; if a medical specialist is not required then determining if medications are required 1306; if medications are not required then printing out the treatment regimen 1310; if medications are required then accessing a pharmaceutical database 1307 to determine which medications are the most beneficial drug or drugs available to treat the diagnosed illness, malady, disease, infection, condition or trauma; printing out the treatment regimen with medications 1308; if a prescription is required 1309 then print out the prescription 1311; then storing patient data and closing patient files 1312, according to some example embodiments.

A method 1400 is described with reference to FIG. 14. In some sample embodiments, FIG. 14 is a diagram of a method for a universal medical diagnostic system which is a continuation of the diagnostic mode that includes determining whether the diagnostic result is unique or a high confidence diagnosis 1402 and if so it proceeds to FIG. 12 (F) 1403 to determine the appropriate treatment regimen; if the diagnostic result is not a unique or high confidence diagnosis, then a determination is made as to whether additional testing would produce an unambiguous or high confidence result 1404 and if so, additional tests are identified and run 1405, test results are received, processed, updated and stored 1406, and proceeds to FIG. 9 (E) 1407; if additional testing is not indicated then a determination is made as to whether medical specialist is required 1408 and if so, identifying a medical specialist 1409 and making a referral 1410; if a medical specialist is not required then referring to a medical doctor for a resolution 1411; disconnecting all sensors and data connections 1412; storing patient data and closing patient files 1413, according to some example embodiments.

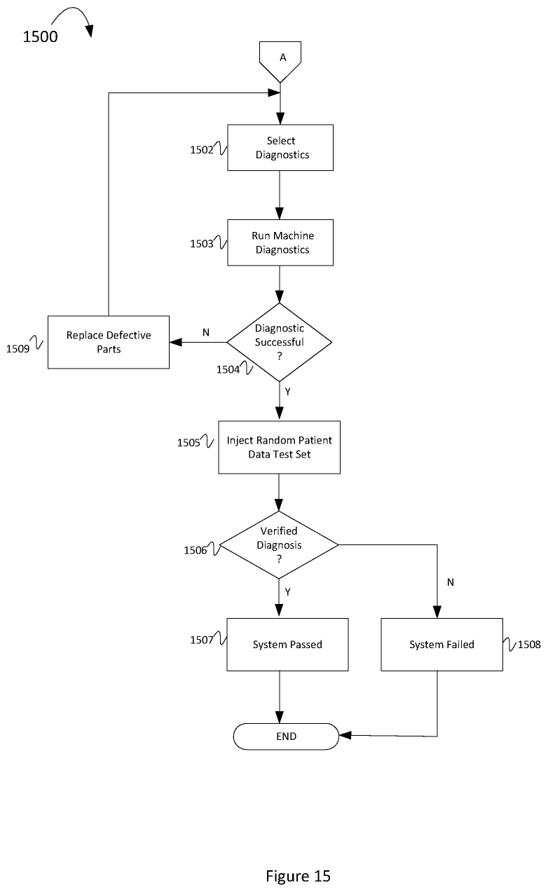

A method 1500 is described with reference to FIG. 15. In some sample embodiments, FIG. 15 is a diagram of a method for a Universal Medical Diagnostic System maintenance mode that includes selecting the machine diagnostics to be run 1502; running the selected machine diagnostics 1503; determining if the diagnostics were successfully completed 1504; replacing the defective part if not 1509; injecting a random patient data test set 1505; verifying the diagnosis 1506; yes system passed 1507; or no system failed 1508.

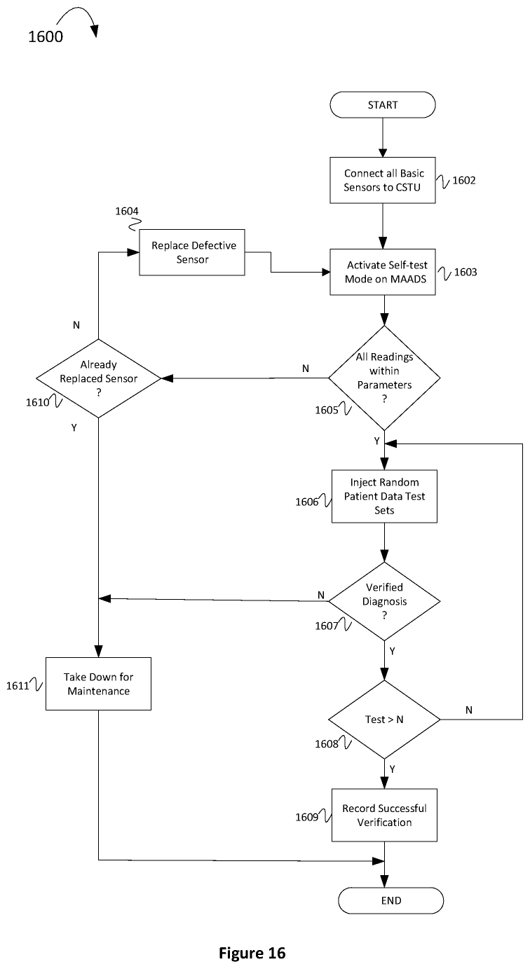

A method 1600 is described with reference to FIG. 16. In some sample embodiments, FIG. 16 is a diagram of a method for a universal medical diagnostic system mode for verification of sensor operation that includes connecting all basic sensors to a certified self test unit 1602; activating the self test mode 1603; determining whether all readings are within preset parameters 1605; if not, determine if sensor already replaced 1610; if no, replace defective sensor 1604; and activate the self test mode 1603; if yes, take the system down for maintenance 1611. If all readings are within preset parameters 1605; inject random patient data test sets 1606; verified diagnosis 1607 failed, take down for maintenance; verified diagnosis 1607 passed, continue injecting random patient data test sets until N (e.g., 50) sets have passed 1608; if N random patient data test sets have passed, record a successful verification 1609, according to some example embodiments. This self test mode will further minimize or eliminate minimize or eliminate potential misdiagnosis or other diagnostic errors.

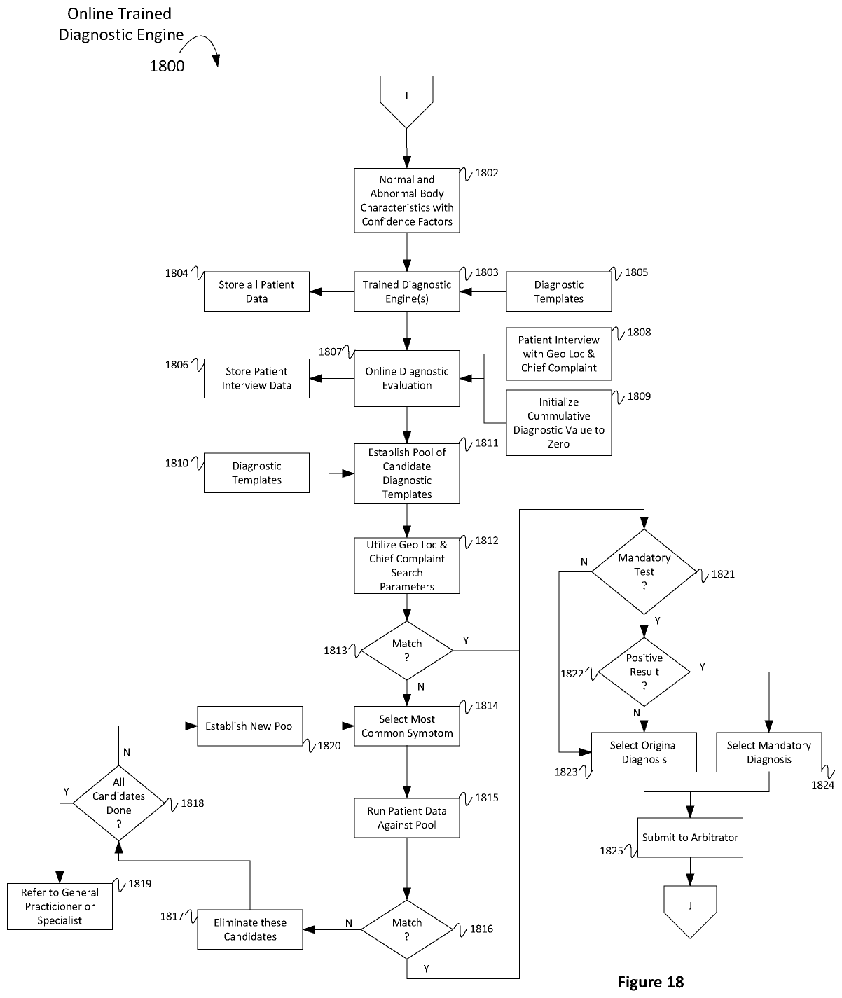

A method 1700 is described with reference to FIG. 17. In some sample embodiments, FIG. 17 is a diagram of a method for a universal medical diagnostic system mode for the classifier portion of the diagnostic engine that includes running all multimedia sensor data through the trained classifier(s) 1703; utilizing multimedia decision 1702 trees and patient's current body characteristics 1704 and storing the patient's current body characteristics to produce both normal and abnormal body characteristics with confidence factors 1705; utilizing a secondary classifier 1707 that is tuned to recognize the patient's historical body characteristics 1706; compare them to the patients current body characteristics 1708; and produce both normal and abnormal body characteristics with confidence factors 1709; before proceeding to FIG. 18 (I) 1802, according to some example embodiments.

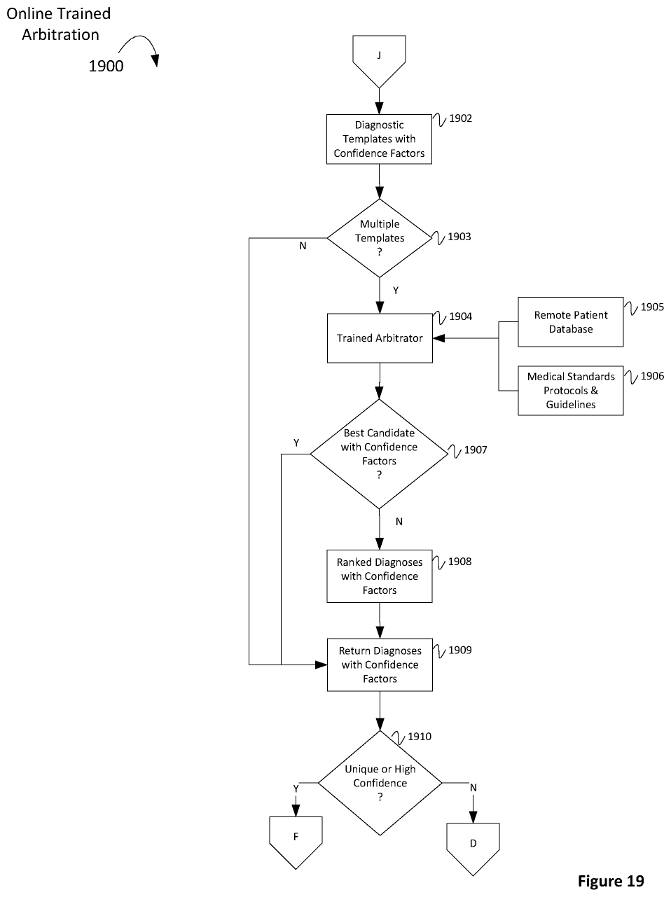

A method 1800 is described with reference to FIG. 18. In some sample embodiments, FIG. 18 is a diagram of a method for a Universal Medical Diagnostic System mode for one of the diagnostic engine portion(s) of the diagnostic engine that includes accepting all processed and available normal and abnormal body characteristics with confidence factors from the classifier section 1802; utilizing the trained diagnostic engine(s) 1803 to store all patient data 1804 and access the appropriate diagnostic templates 1805 before beginning the online diagnostic evaluation 1807, which gathers information related to the geographic location, patient interview and Chief Complaint 1808, stores that data 1806 and initializes the cumulative diagnostic values to zero 1809; it then establishes an initial pool of candidate diagnostic templates 1811 from the Diagnostic Template database 1810 based upon initial search values associated with the geographic location and Chief Complaint; the test pool of candidate templates is searched utilizing the geographic location and Chief Complaint search parameters 1812; if there is a match 1813 then proceed to 1821; if there is no match 1813 then select the most common symptom in all of the templates 1814 and run the patient search parameters and common symptom against the test pool 1815 and check for a match 1816; if there is no match, the test pool of candidates is eliminated 1817 and a test is made to see if the resulting candidate pool is empty 1818; if the initial pool is empty, then refer the patient to a doctor or specialist 1819; if the initial pool is not empty, then establish a new pool using the next most common symptom in the remaining pool 1820 and go to 1814 to continue the loop until a match is found or there are no more candidates; if a match is found on 1813, then check for mandatory tests 1821 and if no mandatory tests then the original diagnosis is selected 1823 and submitted to the arbitrator 1825 before proceeding to FIG. 19 (J) 1902; otherwise if 1821 is positive then the mandatory tests are run 1822; if the test results are negative, then the original diagnosis is submitted to the arbitrator 1825; if the test results are positive then the mandatory test diagnosis is selected 1824 and submitted to the arbitrator 1825 before proceeding to FIG. 19 (J) 1902, according to some example embodiments.

A method 1900 is described with reference to FIG. 19. In some sample embodiments, FIG. 19 is a diagram of a method for a universal medical diagnostic system mode for the arbitrator portion of the diagnostic engine that includes receiving one or more diagnostic templates with confidence factors 1902; if there is only a single template 1903, pass that template with confidence factors to 1909; if there are multiple templates 1903, then submit them to the trained arbitrator 1904 which utilizes a remote patient database 1905 and medical standards, protocols and guidelines 1906 to test for the best candidate template based on confidence factors 1907 and if the result of the test is positive, proceed to return the best candidate diagnoses with confidence factors 1909; if the result of the test is negative, rank all potential diagnoses with confidence factors 1908 and return the ranked diagnoses with confidence factors 1909; if there is no unique or high confidence template, go to FIG. 14 (D); if there is a unique or high confidence diagnosis, go to FIG. 13 (F).

In the foregoing description, numerous specific details such as logic implementations, opcodes, means to specify operands, resource partitioning, sharing, and/or duplication implementations, types and interrelationships of system components, and logic partitioning/integration choices are set forth in order to provide a more thorough understanding of the present invention. It will be appreciated, however, by one skilled in the art that embodiments of the invention may be practiced without such specific details. In other instances, control structures, gate level circuits and full software instruction sequences have not been shown in detail in order not to obscure the embodiments of the invention. Those of ordinary skill in the art, with the included descriptions will be able to implement appropriate functionality without undue experimentation.

References in the specification to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Embodiments of the invention include features, methods or processes that may be embodied within machine-executable instructions provided by a machine-readable medium. A machine-readable medium includes any mechanism which provides (i.e., stores and/or transmits) information in a form accessible by a machine (e.g., a computer, a network device, a personal digital assistant, manufacturing tool, any device with a set of one or more processors, etc.). In example embodiments, a machine-readable medium includes volatile and/or non-volatile media (e.g., read only memory (ROM), random access memory (RAM), magnetic disk storage media, optical storage media, flash memory devices, etc.).

Such instructions are utilized to cause a general purpose or special purpose processor, programmed with the instructions, to perform methods or processes of the embodiments of the invention. Alternatively, the features or operations of embodiments of the invention are performed by specific hardware components which contain hard-wired logic for performing the operations, or by any combination of programmed data processing components and specific hardware components. Embodiments of the invention include software, data processing hardware, data processing system-implemented methods, and various processing operations, further described herein.

In view of the wide variety of permutations to the embodiments described herein, this detailed description is intended to be illustrative only, and should not be taken as limiting the scope of the invention. What are claimed as the invention, therefore, are all such modifications as may come within the scope and spirit of the following claims and equivalents thereto. Therefore, the specification and drawings are to be regarded in an illustrative rather than a restrictive sense.

What is claimed is:

1. A method for controlling operations of a computer system for providing medical diagnoses, comprising:
diagnosing a patient, wherein the diagnosing comprises:
receiving a patient identification of the patient;
receiving electronic data indicating the geographic location of the computer system;
receiving the patient's chief complaint and interview data;
receiving electronic data indicating one or more current body characteristics of the patient comprising at least one of pulse rate, body temperature, blood pressure, respiration, and skin condition;
creating a current multimedia representation for each of the one or more current body characteristics determined by using the one or more sensors;
identifying, in the computer system, previous multimedia representations of each of the one or more body characteristics from other persons using one or more trained classifiers;
comparing the current multimedia representation to the previous multimedia representations;
identifying potential matches with corresponding confidence factors in accordance with defined medical standards;
using one or more computerized trained diagnostic engines of the computer system with diagnostic templates for a set of known illnesses, maladies, diseases, infections, conditions or traumas along with their associated data, signs and symptoms based in part upon geographic location and the patient's chief complaint to select a diagnosis and a diagnosis confidence factor for the patient based on comparing the current multimedia representation to a previous number of multimedia representations derived from previous patients of each of one or more body characteristics in accordance with defined medical standards;
determining that the diagnosis is of highest probability of being the correct diagnosis, using a computerized trained arbitrator, from the one or more computerized trained diagnostic engines of the computer system, in accordance with defined medical standards and in response to the diagnosis confidence factor of the diagnosis exceeding a confidence factor threshold;
in response to the diagnosis confidence factor not exceeding the confidence factor threshold, selecting a different current body characteristic of the patient to determine to increase the diagnosis confidence factor, wherein selection of the different body characteristic is based on the one or more current body characteristics of the patient previously determined and based on an order of selection in order to exceed the high confidence factor threshold with a minimum number of additional selections of different body characteristics; and
in response to the diagnosis confidence factor exceeding the confidence factor threshold, selecting the diagnosis as the diagnosis for the patient; and
selecting a treatment based on the diagnosis.

2. The computerized method of claim 1, wherein diagnosing the patient comprises using multiple differently tuned trained classifiers to optimize the recognition of multimedia patient data sources with corresponding diagnosis confidence factors in accordance with defined medical standards.

3. The computerized method of claim 1, wherein diagnosing the patient comprises using multiple trained diagnostic engines that use diagnostic templates created in accordance with defined medical standards, and
creating the diagnosis confidence factors based on the diagnostic templates.

4. The computerized method of claim 1, wherein diagnosing the patient comprises associating patient's pulse waveform recognition with diagnosis confidence factors in accordance with defined medical standards.

5. The computerized method of claim 1, wherein diagnosing the patient comprises downloading remote patient data from at least one of a local server and a remote server based on an identification of the patient, and wherein selecting the diagnosis and the diagnosis confidence factor for the diagnosis is based at least in part on the remote patient data.

6. The computerized method of claim 1, wherein diagnosing the patient comprises: retrieving historical data of the patient that comprises past body characteristics of the patient that were determined at a prior time, wherein the past body characteristics of the patient comprises at least one of pulse rate, body temperature, blood pressure, respiration, and skin condition, and
wherein selecting the diagnosis and the diagnosis confidence factor for the patient is based at least in part on the historical data of the patient.

7. The computerized method of claim 1, wherein diagnosing the patient comprises:
determining whether an illness, malady, disease, infection, allergic reaction or condition of the patient corresponds to one or more known side effects of or interaction with one or more current medications of the patient.

8. One or more non-transitory machine-readable storage media comprising instructions, that when executed by a processor, cause the processor to perform operations, the instructions comprising:
instructions to diagnose a patient, wherein the instructions to diagnose comprises,
instructions to receive electronic data indicating the geographic location of the computer system;
instructions to receive a patient identification of the patient;
instructions to receive the patient's chief complaint and interview data;
instructions to perform the following instructions until determining that the diagnosis is of highest probability of being the correct diagnosis,
instructions to determine, using one or more body measurement sensors, one or more current body characteristics of the patient comprising at least one of pulse rate, body temperature, blood pressure, respiration, and skin condition;
instructions to create a current multimedia representation for each of the one or more current body characteristics determined by using the one or more body measurement sensors;
instructions to identify previous multimedia representations of each of the one or more body characteristics from other persons using one or more trained classifiers;
instructions to compare the current multimedia representation to previous multimedia representations of each of the one or more body characteristics from other persons using one or more trained classifiers;
instructions to identify potential matches with corresponding confidence factors in accordance with defined medical standards;
instructions to use one or more computerized trained diagnostic engines with electronic diagnostic templates of the computer system for a set of known illnesses, maladies, diseases, infections, conditions or traumas along with their associated data, signs and symptoms to select a diagnosis and a diagnosis confidence factor for the patient based on comparing the current multimedia representation to a previous number of multimedia representations derived from previous patients of each of one or more body characteristics in accordance with defined medical standards;
instructions to determine that the diagnosis is of highest probability of being the correct diagnosis, using a computerized trained arbitrator of the computer system, from the one or more computerized trained diagnostic engines in accordance with defined medical standards and in response to the diagnosis confidence factor of the diagnosis exceeding a confidence factor threshold;
in response to the diagnosis confidence factor not exceeding the confidence factor threshold, instructions to select a different body characteristic of the patient to determine to increase the diagnosis confidence factor, wherein selection of the different body characteristic is based on the one or more current body characteristics of the patient previously determined and based on an order of selection in order to exceed the high confidence factor threshold with a minimum number of additional selections of different body characteristics, and;
in response to the diagnosis confidence factor exceeding the confidence factor threshold, instructions to select the diagnosis as the diagnosis for the patient; and
instructions to select a treatment based upon the diagnosis.

9. The one or more non-transitory machine-readable storage media of claim 8, wherein the instructions to diagnose the patient comprise instructions to use multiple differently tuned trained classifiers to optimize the recognition of multimedia patient data sources with corresponding diagnosis confidence factors in accordance with defined medical standards.

10. The one or more non-transitory machine-readable storage media of claim 8, wherein the instructions to diagnose the patient comprise:
instructions to use multiple trained diagnostic engines that use diagnostic templates created in accordance with defined medical standards; and
instructions to create the diagnosis confidence factors based on the diagnostic templates.

11. The one or more non-transitory machine-readable storage media of claim 8, wherein the instructions to diagnose the patient comprise instructions to associate pulse waveform recognition with diagnosis confidence factors in accordance with defined medical standards.

12. The one or more non-transitory machine-readable storage media of claim 8, wherein the instructions to diagnose the patient comprise instructions to download remote patient data from at least one of a local server and a remote server based on an identification of the patient, and wherein the instructions to select the diagnosis and the diagnosis confidence factor for the diagnosis is based at least in part on the remote patient data.

13. The one or more non-transitory machine-readable storage media of claim 8, wherein the instructions to diagnose the patient comprise:
instructions to retrieve historical data of the patient that comprises past body characteristics of the patient that were determined at a prior time, wherein the past body characteristics of the patient comprises at least one of pulse rate, body temperature, blood pressure, respiration, and skin condition, and
wherein the instructions to select the diagnosis and the diagnosis confidence factor for the patient is based at least in part on the historical data of the patient.

14. The one or more non-transitory machine-readable storage media of claim 8, wherein the instructions to diagnose the patient comprise:
instructions to determine whether an illness, malady, disease, infection or condition of the patient corresponds to one or more known side effects of or interaction with one or more current medications of the patient.

15. An apparatus for controlling operations of a computer system for providing medical diagnoses, comprising:
a processor; and
a machine-readable medium having instructions executable by the processor to cause the apparatus to,
diagnose a patient, wherein the instructions executable by the processor to cause the apparatus to diagnose comprises instructions executable by the processor to cause the apparatus to:
receive electronic data indicating the geographic location of the computer system;
receive a patient identification of the patient;
receive the patient's chief complaint and interview data;
perform the following operations until determining that the diagnosis is of highest probability of being the correct diagnosis,
determine, using one or more body measurement sensors, one or more current body characteristics of the patient comprising at least one of pulse rate, body temperature, blood pressure, respiration, and skin condition;
create, in the computer system, a current multimedia representation for each of the one or more current body characteristics determined by using the one or more body measurement sensors;
identify, in the computer system, previous multimedia representations of each of the one or more body characteristics from other persons using one or more trained classifiers based in part upon the geographic location and the patient's Chief Complaint;
compare the current multimedia representation to the previous multimedia representations of each of the one or more body characteristics from other persons using one or more trained classifiers;
identify potential matches with corresponding confidence factors in accordance with defined medical standards;
use one or more computerized trained diagnostic engines of the computer system with electronic diagnostic templates for a set of known illnesses, maladies, diseases, infections, conditions or traumas along with their associated data, signs and symptoms to select a diagnosis and a diagnosis confidence factor for the patient based on comparing the current multimedia representation to a previous number of multimedia representations derived from previous patients of each of one or more body characteristics in accordance with defined medical standards;
determine that the diagnosis is of highest probability of being the correct diagnosis, using a computerized trained arbitrator, from the one or more computerized trained diagnostic engines in accordance with defined medical standards and in response to the diagnosis confidence factor of the diagnosis exceeding a confidence factor threshold;
in response to the diagnosis confidence factor not exceeding the confidence factor threshold, select a different body characteristic of the patient to determine to increase the diagnosis confidence factor, wherein selection of the different body characteristic is based on the one or more current body characteristics of the patient previously determined and based on an order of selection in order to exceed the high confidence factor threshold with a minimum number of additional selections of different body characteristics, and;
in response to the diagnosis confidence factor exceeding the confidence factor threshold,
select the diagnosis as the diagnosis for the patient; and
select a treatment based upon the diagnosis.

16. The apparatus of claim 15, wherein the instructions executable by the processor to cause the apparatus to diagnose comprises instructions executable by the processor to cause the apparatus to:
use multiple differently tuned trained classifiers to optimize the recognition of multimedia patient data sources with corresponding diagnosis confidence factors in accordance with defined medical standards.

17. The apparatus of claim 15, wherein the instructions executable by the processor to cause the apparatus to diagnose comprises instructions executable by the processor to cause the apparatus to:
use multiple trained diagnostic engines that use diagnostic templates created in accordance with defined medical standards, and
create the diagnosis confidence factors based on the diagnostic templates.

18. The apparatus of claim 15, wherein the instructions executable by the processor to cause the apparatus to diagnose comprises instructions executable by the processor to cause the apparatus to associate pulse waveform recognition with diagnosis confidence factors in accordance with defined medical standards.

19. The apparatus of claim 15, wherein the instructions executable by the processor to cause the apparatus to diagnose comprises instructions executable by the processor to cause the apparatus to download remote patient data from at least one of a local server and a remote server based on an identification of the patient, and wherein the instructions to executable by the processor to cause the apparatus to select the diagnosis and the diagnosis confidence factor for the diagnosis is based at least in part on the remote patient data.

20. The apparatus of claim 15, wherein the instructions executable by the processor to cause the apparatus to diagnose comprises instructions executable by the processor to cause the apparatus to:
retrieve historical data of the patient that comprises past body characteristics of the patient that were determined at a prior time, wherein the past body characteristics of the patient comprises at least one of pulse rate, body temperature, blood pressure, respiration, and skin condition, and
wherein the instructions executable by the processor to cause the apparatus to select the diagnosis and the diagnosis confidence factor for the patient is based at least in part on the historical data of the patient.
